(19) United States
(12) Patent Application Publication
(10) Pub. No.: US 2004/0035690 A1
(43) Pub. Date: Feb. 26, 2004

(54) Method and apparatus for chemical and biochemical reactions using photo-generated reagents

(75) Inventor: Erdogan Gulari, Ann Arbor, MI (US)

Correspondence Address: MEDLEN & CARROLL, LLP, Suite 350, 101 Howard Street, San Francisco, CA, 94105, US

(73) Assignee: The Regents Of The University Of Michigan

(21) Appl. No.: 10/285,824

(22) Filed: Nov. 1, 2002

Related U.S. Application Data
(63) Continuation-in-part of application No. 10/157,442, filed on May 29, 2002.
Application No. 10/157,442 is a continuation of application No. 09/248,093, filed on Feb. 10, 1999, now Pat. No. 6,426,184.
(60) Provisional application No. 60/074,368, filed on Feb. 11, 1998.

(51) Int. Cl. C07C 5/00

(52) U.S. Cl. 204/157.15; 204/157.61

(57) ABSTRACT

This invention provides method and apparatus for performing chemical and biochemical reactions in solution using in situ generated photo-products as reagent or co-reagent. Specifically, the method and apparatus of the present invention have applications in parallel synthesis of molecular sequence arrays on solid surfaces.

This application is a continuation-in-part that claims the benefit of U.S. application Ser. No. 10/157,442 filed May 29, 2002 which is a continuation of U.S. Pat. No. 6,426,184 filed on Feb. 10, 1999 which claims the benefit of U.S. Provisional Application No. 60/074,368, filed on Feb. 11, 1998.

### FIELD OF INVENTION

The present invention relates to parallel synthesis and assay of biopolymers on a substrate surface. Specifically, the present invention relates to the parallel synthesis and assay of biopolymers using light-directed generation of photo-generated reagents. Even more specifically, the present invention relates to the parallel synthesis and assay of biopolymers using light-directed generation of photo-generated reagents on microarray biochips.

### BACKGROUND

The rapid development of modern medicine, agriculture, and materials has imposed enormous demands on technological and methodological progress to accelerate sample screening in chemical and biological analysis. Development of parallel processes on a micro-scale is one technological field that may have potential in solving some of the current problems. Many advances have been made in this area using parallel synthesis, robotic spotting, inkjet printing, and microfluidics. Marshall et al., Nature Biotech. 16, 27-31 (1998) Continued efforts, however, are necessary to establish more reliable, flexible, faster, and inexpensive technologies.

High-throughput screening applications have been modified to utilize molecular microarray (MMA) biochips. Specifically, these biochips contain high-density arrays of biopolymers immobilized on solid surfaces. These biochips have been reported useful for exploring molecular genetic and sequence information. Marshall et al., Nature Biotech. 16, 27-31 (1998); Ramsay, Nature Biotech. 16, 40-44 (1998) Specifically, target molecules have been hybridized to DNA oligonucleotides and cDNA probes on biochips for determining nucleotide sequences, probing multiplex interactions of nucleic acids, identifying gene mutations, monitoring gene expression, and detecting pathogens. Schena et al., Science 270, 467-460 (1995); Lockhart et al., Nature Biotech. 14, 1675-1680; Weiler, Nucleic Acids Res. 25, 2792-2799 (1997); de Saizieu et al., Nature Biotech. 16, 45-48; and Drmanac et al., Nat. Biotechnol., 16, 54-58 (1998).

Biochip fabrication processes include: i) direct on-chip synthesis (making several sequences at a time) using inkjets, ii) direct on-chip parallel synthesis (making the whole array of sequences simultaneously) using photolithography, and iii) immobilization of a library of presynthesized molecules using robotic spotting (Ramsay, Nature Biotech. 16, 40-44 (1998)). These current methods of making oligopeptide libraries with a microarray platform rely on amino acids protected with photolabile group and several photomask plates to control the peptide synthesis. This approach is considered to be inconvenient and expensive because those amino acids are not commercially available and many photomask plates need to be prepared. Additional limitations regarding the use of photomasks are: i) the setup for making a new chip is very expensive due to the large number of photomasks that have to be made; ii) photolithography equipment is expensive and, therefore, can not be accessed by many interested users; iii) photolithography processes have to be conducted in an expensive cleanroom facility and require trained technical personnel; and iv) the entire process is complicated and difficult to automate. These limitations undermine the applications of oligonucleotide chips and the development of the various MMA-chips.

Therefore, there is a genuine need for the development of chemical methods and synthesis apparatus that are simple, versatile, cost-effective, easy to operate, and that can afford molecular arrays of improved purity.

### SUMMARY OF INVENTION

The present invention relates to parallel synthesis and assay of biopolymers on a substrate surface. In one embodiment, the present invention relates to the parallel synthesis and assay of biopolymers using light-directed generation of photo-reagents. In another embodiment, the present invention relates to the parallel synthesis and assay of biopolymers using light-directed generation of photo-reagents on microarray biochips.

The present invention provides methods and apparatus for performing chemical and biochemical reactions in solution using in situ generated photo-products as reagents or coreagents. These reactions are controlled by illumination, such as with UV or visible light. Unless otherwise indicated, all reactions described herein occur in solutions of at least one common solvent or a mixture of more than one solvent. The solvent can be any conventional solvent traditionally employed in the chemical reaction, including but not limited to such solvents as CH₂Cl₂, CH₃CN, toluene, hexane, CH₃OH, H₂O, and/or an aqueous solution containing at least one added solute, such as NaCl, MgCl₂, phosphate salts, etc.

In one aspect of the present invention, the solvent solution is contained within defined areas on a solid surface containing an array of reaction sites. Preferably, these reaction sites are isolated and may be, but are not limited to, reaction cartridiges, reaction wells, non-wetting areas and microwells. In one embodiment, a photo-generated reagent (PGR) is formed by applying a solution containing at least one photo-generated reagent precursor (PGRP) on the solid surface and subsequently projecting a controllable light pattern onto the solid surface, at least one PGR forms at illuminated sites; no reaction occurs at dark (i.e., non-illuminated) sites. In another embodiment, PGR-modified reaction conditions may undergo multiple reactions wherein at least one step of a multi-step reaction at a specific isolated reaction site on the solid surface may be controlled by illumination (i.e., light). A further embodiment of the present invention, contemplates parallel reactions, wherein at each step of the reaction only selected isolated reaction sites on a solid surface or array (i.e., microarray or biochip) are allowed to simultaneously react by the present or absence of illumination.

Another aspect of the present invention contemplates an apparatus for performing the light-controlled synthesis of biopolymers using PGRPs. In one embodiment, the present invention contemplates an apparatus that controls the synthesis of biopolymers on a solid surface containing a plurality of isolated reaction sites. Preferably, the apparatus controls the synthesis of biopolymers by a spatially directed light pattern generated using a computer and a digital optical projector, wherein the controlled light pattern is projected onto specific isolated reaction sites.

Another aspect of the present invention contemplates the in situ generation of chemical/biochemical reagents (i.e., for example, PGRs) that are used in the subsequent synthesis of biopolymers at certain selected reaction sites. One embodiment of the present invention contemplates changing the solvent solution pH by controlled photo-generation of acids or bases. Preferably, the pH conditions of selected isolated reaction sites can be independently controlled by adjusting a spatially directed light pattern. In one embodiment, the changes in pH conditions effect chemical or biochemical reactions including, but not limited to activating enzymes, and inducing couplings and cross-linking through covalent or non-covalent bond formation between ligand molecules and their corresponding receptors.

Another aspect of the present invention contemplates the binding of PGRs, with other molecules in solution. In one embodiment, the concentration of PGRs is determined by the dose of illumination (i.e., for example, light) thereby allowing a simultaneous determination of PGR binding affinities and specificities under a plurality of conditions.

Another aspect of the present invention contemplates the parallel synthesis of biopolymers, including, but not limited to, oligonucleotides and peptides, under conditions involving selective deprotection or coupling reactions that result in a controlled fabrication of diverse biopolymers on solid surfaces. In one embodiment, these solid surfaces comprise molecular microarray chips (MMA-chips), microarrays and biochips. In another embodiment, the present invention contemplates the detection and analysis of gene sequences and their interactions with other molecules, including, but not limited to antibiotics, antitumor agents, oligosacchrides, and proteins. In yet another embodiment, a photo-generated reagent acid (acid PGR) is used to remove the protection group from amino acids or peptide oligomers. In one particular embodiment, this acid PGR is trisarylsulfonium antimonyhexafluoride (SSb). In another embodiment, the acid PGRs are generated from acid PGRPs in situ by a controlled light pattern projected on a solid substrate comprising isolated reaction sites. Preferably, a maskless laser light illumination system is used to generate acid PGRs from the acid PGRPs.

Another aspect of the present invention contemplates verifying the accuracy of the biopolymer sequence. In one embodiment, an amino acid sequence of the synthesized oligopeptide and the location of its synthesis is verified by specific recognition binding. In another embodiment, the specific recognition binding is performed by models selected from the group consisting of a lead (II) (i.e., Pb²⁺) ion-peptide biosensor for Pb²⁺ and human protein p53 detection by, including but not limited to, residues 20-25 of mouse MAb DO1.

In another embodiment, the current invention contemplates identifying the correct locations of biopolymer synthesis on the microarray by fluoresence labeling. Preferably, synthesized peptides having a predetermined sequence, and their analogues, have their locations correctly idenfified on a microarray by specific binding treatment and fluorescence labeling, wherein the fluorescence emission images of the oligopeptide microarray show variable fluorescence intensity. In another embodiment, the stepwise synthesis efficiencies of pentapeptide synthesis on the microwell substrate range are approximately 96-100% and do not decrease with respect to the chain length of the peptide.

One aspect of the present invention contemplate a method, comprising: a) providing; i) a plurality of protected polypeptides attached to a substrate, each of said protected polypeptides comprising at least one protecting group, said substrate configured so as to comprise of a plurality of isolated reaction sites; ii) a mixture comprising triarylsulfonium antimonyhexafluoride; and iii) a laser; b) contacting one or more of said isolated reaction sites with said mixture to create one or more contacted reaction sites; and c) illuminating one or more said contacted reaction sites with said laser under conditions such that said protecting group of at least one protected polypeptide is removed so as to create a deprotected polypeptide. In one embodiment, the mixture further comprises dichloromethane. In another embodiment, the mixture further comprises perylene. In one embodiment, the substrate prior to step (a) is derivatized. In one specific embodiment, the substrate is derivatized with triethoxysilane. In one embodiment, the protecting group is tert-butoxycarbonyl. In an alternative embodiment, the present invention further comprising step d) contacting said deprotected polypeptide with a first protected monomer under conditions such that said first protected monomer is coupled to said polypeptide to create a first extended polypeptide, said first protected monomer comprising a protecting group. In another embodiment, the present invention further comprising step e) illuminating said first extended polypeptide with said laser under conditions such that said protecting group of said first protected monomer is removed so as to create a second extended polypeptide. In yet another embodiment, the present invention further comprising step (f) contacting said second extended polypeptide with a second protected monomer under conditions such that said second protected monomer is coupled to said second extended polypeptide to create a third extended polypeptide, said third extended polypeptide comprising a protecting group. In one embodiment, the plurality of isolated reaction sites of step (a) is selected from the group consisting of reaction wells, reactor assembly cartidges and non-wetting surfaces.

Another aspect of the present invention contemplate a method, comprising: a) providing; i) a plurality of protected first monomers attached to a substrate, each of said protected first monomers comprising at least one protecting group, said substrate configured so as to comprise of a plurality of isolated reaction sites; ii) a mixture comprising trisarylsulfonium antimonyhexafluoride; and iii) a laser; b) contacting one or more of said isolated reaction sites with said mixture to create one or more contacted reaction sites; and c) illuminating one or more said contacted reaction sites with said laser under conditions such that said protecting group of at least one protected first monomer is removed so as to create a deprotected first monomer. In one embodiment, the mixture further comprises dichloromethane. In another embodiment, the mixture further comprises perylene. In one embodiment, the substrate prior to step (a) is derivatized. In one specific embodiment, the substrate is derivatized with triethoxysilane. In one embodiment, the protecting group is tertbutoxycarbonyl. In one embodiment, the present invention further comprising step d) contacting said deprotected first monomer with a protected second monomer under conditions such that said protected second monomer is coupled to said first monomer to create a first protected biopolymer, said protected second monomer comprising a protecting group. In another embodiment, the present invention further comprising step e) illuminating said first protected biopolymer with said laser under conditions such that said protecting group of said protected second monomer is removed so as to create a first deprotected biopolymer. In yet another embodiment, the present invention further comprising step (f) contacting said first deprotected biopolymer with a third protected monomer under conditions such that said third protected monomer is coupled to said first deprotected biopolymer to create a second protected biopolymer, said second protected biopolymer comprising a protecting group. In one embodiment, said monomer comprises an amino acid.

Another aspect of the present invention contemplates, a method, comprising: a) providing; i) a plurality of protected linkers attached to a substrate, each of said protected linkers comprising at least one protecting group, said substrate configured so as to comprise of a plurality of isolated reaction sites; ii) a mixture comprising triarylsulfonium antimonyhexafluoride; and iii) a laser; b) contacting one or more of said protected linkers with said mixture to create one or more contacted protected linkers; and c) illuminating one or more said protected linkers with said laser under conditions such that said protecting group of at least one of said protected linkers is removed so as to create a deprotected linker. In one embodiment, the mixture further comprises dichloromethane. In another embodiment, the mixture further comprises perylene. In one embodiment, the substrate prior to step (a) is derivatized. In one specific embodiment, the substrate is derivatized with triethoxysilane. In one embodiment, the protecting group is tert-butoxycarbonyl. In one embodiment, the present invention further comprising step d) contacting said deprotected linker with a first protected monomer under conditions such that said first protected monomer is coupled to said deprotected linker to create a first multimer, said first protected monomer comprising a protecting group. In another embodiment, the present invention further comprising step e) illuminating said first multimer with said laser under conditions such that said protecting group of said first protected monomer is removed so as to create a deprotected first multimer. In yet another embodiment, the present invention further comprising step (f) contacting said deprotected first multimer with a second protected monomer under conditions such that said second protected monomer couples with said deprotected first monomer to create a second multimer, said second protected monomer comprising a protecting group.

Another aspect of the present invention contemplates a method comprising: a) providing; i) a plurality of polypeptides attached to a solid substrate; and ii) a cation capable of fluoresence emission; b) contacting said polypeptides with a fluid comprising said cation; and c) detecting said polypeptides by fluoresence emission intensity. In one embodiment, said cation comprises Pb²⁺. In another embodiment said cation comprises As³⁺. In one embodiment said polypeptide comprises dns-Glu-Cys-Glu-Glu. In another embodiment said polypeptide comprises dns-Glu-Glu-Glu-Glu.

Obviously, the methods described above are capable of producing any desired length of any desired sequence and composition of any desired monomers.

Definitions

As used herein the term “photo-generated reagent precursor” includes any compound that either forms at least one intermediate or product upon “illumination” or becomes active upon illumination. Such “illumination” includes, but is not limited to, any wavelength of light (i.e., for example, ultraviolet) from any light source (e.g., incandescent, fluorescent, phosphorescent, and laser).

As used herein the term “photo-generated reagent” includes any compound that is capable of initiating a chemical or photochemical reaction such that deprotection occurs.

As used herein the term “solid substrate” includes any material capable of providing a surface for synthesis of biopolymers. For example, a solid substrate may comprise isolated reaction sites capable of attaching a “linker”.

As used herein the term “linker” includes any molecule capable of attaching to a solid substrate, either covalently or non-covalently, wherein functional groups may bind with protecting agents or biopolymer monomers. It is not contemplated that linkers include amino acids, nucleotides or saccharides.

As used herein the term “biopolymer” includes any organic chain of monomer units including, but not limited to, proteins, oligonucleotides, oligodeoxynucleotides, oligopolysaccharides, and oligoribodeoxynucleotides.

As used herein the term “monomer” includes any organic molecule that when bound to other monomers of similar chemical composition create a biopolymer. Such monomers include, but are not limited to, amino acids, nucleosides, deoxynucleosides, ribodeoxynucleosides and saccharides.

As used herein the term “reaction site” includes any location on a solid substrate wherein a linker is attached. A reaction site may or may not be “isolated” (i.e., separated from other sites, whether by walls etc., or by spacing) wherein such isolated reaction sites include, but are not limited to, reaction wells, microarray microwells, reaction cartridges, and non-wetting surfaces.

As used herein the term “reactive functional group” refers to a reactive moiety of any molecule that is not involved bonding, either covalently or non-covalently: i) a linker to a solid surface, ii) a first monomer to a linker, or iii) a second, or subsequent monomer to a multimer. A reactive functional group preferably binds protecting groups to prevent branching during the synthesis process.

As used herein the term “reactive terminal group” refers to a reactive moiety of any molecule that is involved in bonding, either covalently or non-covalently; i) a linker to a solid surface, ii) a first monomer to a linker, or iii) a second, or subseuent monomer to a multimer. A reactive terminal group includes, but is not limited to a hydroxyl group.

As used herein the term “environment” includes the internal volume of an isolated reaction site, wherein said internal volume consists of a fluid.

As used herein the term “light pattern” includes any maskless controlled display of illumination onto a solid surface. A “controlled light pattern” may be generated by a computer in combination with a spatial optical modulator that comprises any variation of light and dark areas.

As used herein the term “spatial optical modulator” includes any device capable of producing a controlled light pattern. A spatial optical modulator includes, but is not limited to, a digital micromirror device, a reflective liquid crystal display device and a transmissive liquid crystal display device.

As used herein the term “protecting group” includes any compound capable of bonding to a reactive terminal group or a reactive functional group on a linker or monomer. A protecting group includes, but is not limited to butyloxylcarbonyl (Boc) and fluoroenylmethyloxycarbonyl (Fmoc). In addition, a protecting group may be either “acid-labile” or base-labile” wherein, respectively a decrease or increase in the pH of the environment breaks the bond between the protecting group and the functional group.

As used herein the term “photo-generated activator” includes any compound bound to a monomer that is released from the monomer by illumination subsequent to the monomer bonding to a biopolymer or linker attached at a reaction site.

As used herein the term “extended polypeptide” refers to any sequence of amino acids that is the product of photo-generated reagent coupling.

As ussed herein the term “multimer” refers to any sequence of monomers that are coupled to a linker.

As used herein the term “mixture” refers to combinations of at least two compounds, including but not limited to a compound dissolved in a solvent.

These and other aspects demonstrate features and advantages of the present invention. Further details are made clear by reference to the remaining portions of the specification and the attached drawings.

### BRIEF DESCRIPTION OF THE DRAWINGS

FIG. 1 is a drawing of oligonucleotide synthesis using photo-generated acids. L=linker group; Pₐ=acid-labile protecting group; H⁺=photo-generated acid; T, A, C, and G=nucleotide phosphoramidite monomers; hv=light exposure.

FIG. 2 is a drawing of the deprotection process using photo-generated acids in oligonucleotide synthesis.

FIG. 3 is a drawing of oligonucleotide synthesis using photo-generated reagents. The process is the same as shown in FIG. 1 except that a photo-generated activator, such as dimethoxybenzoinyltetrazole, is used, while the deprotection step is accomplished using a conventional acid.

FIG. 4 is a drawing of amino acid deprotection using photo-generated acids or photogenerated bases. Boc=butyloxylcarbonyl; Fmoc=fluoroenylmethyloxycarbonyl.

FIG. 5 is a drawing of peptide synthesis using photo-generated acids. L=linker group; Pₐ=acid-labile protecting group; F, Q, D, Y, S, and A=representative Boc-protected amino acids; hv=light exposure.

FIG. 6 is a drawing of peptide synthesis using photo-generated bases. L=linker group; P_{b}=base-labile protecting group; F, Q, D, Y, S, and A=representative Fmoc-protected amino acids; hv=light exposure.

FIG. 7 is a drawing of oligopolysacharride synthesis using both photo-generated acids and photo-generated bases at various of reaction steps.

FIG. 8A is a schematic illustration of the synthesis apparatus using a micromirror array device.

FIG. 8B is a schematic illustration of the synthesis apparatus using a reflective LCD array device.

FIG. 8C is a schematic illustration of the synthesis apparatus using a transmissive LCD array device.

FIG. 9A illustrates one embodiment of isolated reaction sites using a microwell cover.

FIG. 9B illustrates one embodiment of isolated reaction sites using a microwell solid substrate.

FIG. 9C illustrates one embodiment of isolated reaction sites using a patterned non-wetting film on a solid substrate.

FIG. 10 is an exploded schematic of one embodiment of a reaction cartridge exemplifying an enlarged view of reaction-wells.

FIG. 11A is a schematic illustration of an exemplary deprotection reaction demonstrated using a partially masked reaction-well.

FIG. 11B is a schematic illustration of an exemplary deprotection reaction demonstrated using a reaction-well being partially exposed.

FIG. 12 illustrates one embodiment of a stepping mechanism for parallel synthesis of a plurality of arrays.

FIG. 13 is a plot of H₃O⁺ chemical shift (ppm) versus light illumination time (min) measured from a sample containing an exemplary photo-acid precursor.

FIG. 14 shows exemplary HPLC profiles of DNA (FIG. 14A) and RNA (FIG. 14B) nucleosides deprotected using a photo-generated acid.

FIG. 15 shows exemplary HPLC profiles of DNA oligomers synthesized using a photo-generated acid.

FIG. 16 shows exemplary HPLC profiles of an amino acid deprotected using a photo-generated acid.

FIG. 17A illustrates one embodiement of a fabrication process for making microwells on a solid substrate.

FIG. 17B is an enlarged photograph of exemplary microwells on a solid substrate comprising glass.

FIG. 18A illustrates one embodiment of a fabrication process for making a non-wetting-film pattern on a solid substrate.

FIG. 18B is an enlarged photograph of methanol-droplets formed on a solid substrate comprising glass containing an exemplary patterned non-wetting surface.

FIG. 19 is an exemplary image of fluorescein-tagged thymine attached to a patterned non-wetting surface of a solid substrate comprising glass.

FIG. 20 is a schematic diagram of one embodiment comprising a light illumination setup.

FIG. 21 demonstrates the stepwise efficiency of pentaglycine synthesis. The figure shows one embodiment depicting deprotection using either 50% TFA (open bars) or 10% SSb (filled bars).

FIG. 22 displays a demonstrative microarray of tetrapeptides for Pb²⁺ binding:

Panel A. An exemplary microwell array pattern of localized parallel synthesis of dansyl-labeled tetrapeptide, dns-Glu-Cys-Glu-Glu (open circles), dns-Glu-Glu-Glu-Glu (crosshatched circles), dns-Cys-Cys-Cys-Cys (gray circles) and dns-Gly-Gly-Gly-Gly (filled circles);

Panel B. An exemplary fluorescence image showing selective labeling of dns-Glu-Cys-Glu-Glu with Pb²⁺;

Panel C. An exemplary fluorescence image showing selective labeling of dns-Glu-Glu-Glu-Glu with As³⁺.

FIG. 23 displays a demonstrative microarray of hexapeptides for immunoassay:

Panel A. A sample microwell array pattern of parallel synthesis of Ser-Asp-Leu-His-Lys-Leu (open circles), Asp-Ser-Leu-His-Lys-Leu (gray circles), and Ser-Gly-Leu-His-Lys-Leu (filled circles);

Panel B. An exemplary fluorescence image showing selective labeling of Ser-Asp-Leu-His-Lys-Leu with mouse monoclonal antibody DO-1 directed against amino acid residue 20-25 of human p53 peptide using an FITC-tagged secondary rabbit anti-mouse immunoglobulin (IgG).

FIG. 24 illustrates an exemplary fluorescence emission image of pentaglycine synthesis after each deprotection step using 2.5% SSb mixed with perylene (1:0.1). The labeled values were the average fluorescence intensity and the stepwise synthesis efficiency. The spikes at the edges are due to reflections from the side walls. Actual pixels are shown above.

### DETAILED DESCRIPTION OF INVENTION

The present invention relates to parallel synthesis and assay of biopolymers on a substrate surface. In one embodiment, the present invention relates to the parallel synthesis and assay of biopolymers using light-directed generation of photo-reagents. In another embodiment, the present invention relates to the parallel synthesis and assay of biopolymers using light-directed generation of photo-reagents on microarray biochips.

The present invention contemplates fundamental improvements compared to previous methods for parallel synthesis of biopolymers. Pirrung et al., U.S. Pat. No. 5,143,854 (1992); Fodor et al., U.S. Pat. No. 5,424,186 (1995); and Beecher et al., PCT Publication No. WO 98/20967 (1997). Specifically, the present invention contemplates utilizing existing combinatorial chemistry, wherein at least one of the reagents is replaced with a photo-generated reagent precursor (PGRP). Therefore, unlike previous methods, which require monomers containing photolabile protecting groups or a polymeric coating layer as the reaction medium, the present invention contemplates using monomers developed by conventional combinatorial chemistry that requires minimal variation in conventional synthetic chemistry protocols.

The improvements made possible by the present invention have significant consequence. For example, the synthesis of biopolymers on microarrays can be easily integrated into an automated DNA/RNA synthesizer, thus greatly simplifying the process and reducing the cost. Additionally, conventional chemistry routinely achieves better than 98% yield per step when synthesizing oligonucleotides. This yield is far superior to the 85-95% yield obtained by the previous method of using photolabile protecting groups. Pirrung et al., J. Org. Chem. 60, 6270-6276, (1995); McGall et al., J. Am. Chem. Soc. 119, 5081-5090 (1997); and McGall et al., Proc. Natl. Acad. Sci. USA 93, 13555-13560 (1996). This improved stepwise yield is critical for synthesizing high-quality oligonucleotides using microarrays having diagnostic and clinical applications. Surprisingly, the yield of photo-generated reagents (PGRs) is not a major concern in one embodiment of the present invention in contrast to previous methods that had the disadvantage of incomplete deprotection on photolabile protecting groups. The present invention contemplates a biopolymer synthesis process that can be monitored that is not possible using previous methods.

Other embodiments contemplated by the present invention include an easy expansion to the synthesis of non-conventional biopolymers on microarrays including, but not limited to, oligonucleotides containing modified residues, 3′-oligonucleotides (as opposed to 5′-oligonucleotides obtained in a normal synthesis), peptides, oligosacchrides, combinatory organic molecules, and the like. These undertakings would be extremely difficult using previous methods that require monomers containing photolabile-protecting groups and the development of new synthetic procedures for each monomer type.

The present invention contemplates using modified residues and various monomers that are already commercially available. Specifically, the present invention contemplates application to all types of reactions, including but not limited to biopolymer synthesis and chemical amplification. Additionally, the reaction time for each step of a biopolymer synthesis process using conventional oligonucleotide chemistry (i.e., for example, approximately 5 minutes per step) is much shorter than previous methods using photolabile blocked monomers (i.e., in excess of 15 minutes per step).

The generation of light patterns in previous methods of biochip fabrication use conventional photomask-based lithography tools. Karl et al., U.S. Pat. No. 5,593,839 (1997). A significant drawback of this method is that the number and pattern complexity of the masks increase as the length and sequence variation of the biopolymers increase. For example, a total of 48 masks (i.e., 4×12) are required to synthesize a subset of dodecanucleotides, and this number may be larger depending on the choice of custom chip. Obviously, to make a new set of sequences, a new set of masks have to be prepared. A high degree of difficulty is associated with the required high precision alignment (i.e., on the order of <10 μm resolution) of the successive photomasks; a task that mandates the use of specialized equipment and highly skilled technical expertise. This conventional photomask technology is only semi-automatic, inflexible and expensive. In addition, the conventional photomask fabrication process requires expensive cleanroom facilities and demands special technical expertise in microelectronic fields. Therefore, the entire biochip/microarry fabrication process is inaccessible to most in the research community.

One embodiment of the present invention contemplates replacing conventional photomasks with a computer-controlled spatial optical modulator so that light patterns are generated by a computer in a manner similar to image presentations using a standard digital monitor (i.e., for example, a television or computer screen). This novel approach to controlling light patterns provides maximum flexibility that accomodates any design of microarray. The novel approach also simplifies the fabrication process by eliminating the need for performing mask alignment as in the conventional photolithography, which is time-consuming and prone to alignment errors.

In another embodiment, the present invention contemplates an optical system and a reactor assembly integrated into an apparatus suitable for use in a standard laboratory without the need for a cleanroom. Preferably, an apparatus designed for the standard laboratory is fully controllable by a personal computer to accomodate custom-designed biopolymer sequences and microarrays.

Clear advantages of several specific embodiments contemplated by the present invention include, but are not limited to, i) easy adoption to streamlined production of large quantities of standard biochips or a fixed number of specialized biochips by automated production lines; ii) a significant reduction in the cost of making microarrays and biochips, iii) significantly increasing the accessibility of microarray and biochip technology to the typical research and biomedical community, and iv) overcoming the limitations of previous biopolymer synthesis methods using standard photolithographic methodology.

### I. Methods for Chemical/Biochemical Reactions using Photo-Generated Reagents (PGRs)

Two methods are currently widely accepted by those having skill in the art regarding synthesis of biopolymers. First, regards the use of photolabile-group protected monomers (Pirrung et al., U.S. Pat. No. 5,143,854 (1992); Fodor et al., U.S. Pat. No. 5,424,186 (1995)) and second, the use of conventional chemical amplification chemistry (Beecher et al., PCT Publication No. WO 98/20967 (1997)). Disadvantages of both methods involve repetitive steps of deprotection, monomer coupling, oxidation, and capping that require the manipulation of specially constructed photomasks to selectively block light exposure. Specifically, the photolabile protecting groups are cleaved from growing oligonucleotide molecules in the illuminated areas while in non-illuminated areas the protecting groups on oligonucleotide molecules are not affected. The nascent oligonucleotide molecules are subsequently contacted with a solution containing monomers having a unprotected first reactive center and a second reactive center protected by a photolabile-protecting group. In the illuminated areas, monomers couple via the unprotected first reactive center with the deprotected oligonucleotide molecules. However, in the nonilluminated areas oligonucleotides remain protected and, therefore, no coupling reaction takes place. The resulting oligonucleotide molecules after the coupling are protected by photolabile protecting groups on the second reactive center of the monomer and the process is repeated until all desired oligonucleotides are synthesized.

For the synthesis process involving chemical amplification chemistry, a planar area is spotted with oligonucleotide molecules and is coated with a thin (a few micrometers) polymer or photoresist layer on top of the oligonucleotide molecules. The free end of each oligonucleotide molecule is protected with an acid-labile group. The polymer/photoresist layer contains a photo-acid precursor and an ester (an enhancer), which, in the presence of H⁺, dissociates and forms an acid. During a synthesis process, acids are produced in illuminated areas within the polymer/photoresist layer and acid-labile protecting groups on the ends of the oligonucleotide molecules are cleaved. The polymer/photoresist layer is then stripped using a solvent or a stripping solution to expose the oligonucleotide molecules below. The area is then contacted with a solution containing monomers having a reactive center protected by an acid-labile protecting group. The monomers couple via the unprotected first reactive center only with the deprotected oligonucleotide molecules in the illuminated areas. In the non-illuminated areas, oligonucleotide molecules still have their protection groups on and, therefore, do not participate in coupling reaction. The substrate is then coated with a photo-acid precursor containing polymer/photoresist again. The illumination, deprotection, coupling, and polymer/photoresist coating steps are repeated until desired oligonucleotides are obtained.

There are significant drawbacks in both of the above methods. Primarily, these limitations specifically involve the photolabile-protecting groups: (a) the chemistry used is non-conventional and the entire process is extremely complicated; and (b) the technique suffers from low sequence fidelity due to chemistry complications. Other limitations specifically involving chemical amplification chemistry include: (a) the application of a polymer/photoresist layer that is not suitable for routinely used chemical and biochemical reactions since isolated reaction sites are not present; (b) destructive chemical conditions may be required for pre- and post-heating and stripping of the polymer/photoresist layer causing the decomposition of oligonucleotides; (c) the entire process is labor intensive and difficult to automate due to the requirement for many cycles (i.e., in excess of 80 cycles for synthesis of a 20-mer) of photoresist coating, heating, alignment, light exposure and stripping; and (d) a broad range of biochemical reactions or biological samples to which a photo-generated reagent is applied is not possible since the embedding of biological samples in a polymer/photoresist layer may be prohibitive.

The present invention contemplates methods for using solution-based photochemical reactions involving in situ photo-generated reagents (PGRs). Normally, a conventional chemical/biochemical reaction occurs between at least one reactant (generically denoted as “A”) and at least one reagent (generically denoted as “R”) to give at least one product as depicted below:

A+R−>A′+R

However, the present invention contemplates reaction conditions that are controlled by illumination, for example, as with light wherein R is produced from a photo-generated reagent precursor (PGRP) and is referred to herein as a photo-generated reagent (PGR). The PGR functions the same as a conventional reagent and the reaction proceeds normally. For example, the overall PGRP—>PGR conversion is depicted below by:

$$\begin{matrix}( PGR Precursor ) {⟶}^{hv} R \\ A + R \to A ' + R '\end{matrix}$$

Some embodiments of the present invention contemplate PGRPs that yield H⁺ in the form of R₁CO₂H, R₁PO₃H, R₁SO₃H, H⁺X⁻ (where R₁═H, alkyl (C₁-Cl₂), aryl (i.e., aromatic structures containing phenyl), or their substituted derivatives; for example, halogen atoms, NO₂, CN, OH, CF, C(O)H, C(O)CH₃, C(O)R₂, SO₂CH₃, SO₂R₂, OCH₃, OR₂, NH₂, NHR₂, NR₂R₃ (where R₂ and R₃=alkyl or aryl (i.e., C₁-C₁₂) and X=halogen atoms or inorganic salt ions, or the like. (see Table 1) Photo-generated acids may also be complexes, such as MₘXₙ (i.e., Lewis acids), where m and n are the number of atoms formed upon illumination. Alternatively, other embodiments of the present invention contemplate PGRPs that yield a base, such as an amine, an oxide or the like, upon illumination. (see Table 1)

TABLE 1A
Examples of Photo-Generated Reagent Precursors and Their Products

| Photo-Generated | | Reagent |
|---|---|---|
| Precursor | Chemical Structure | Generated |
| diazonium salts | 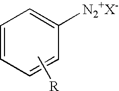 | B(R₁)₃, Al(R₁)₃ |
| | X = B(R₁)₄, AI(R₁)₄ (R₁ = halogen); R = H, halogen, NO₂, CN, | |
| | SO₂R₅, OH, OCH₃, SCH₃, CF₃, OR₅, SR₅, CH₃, t-butyl, C₁-C₁₂- | |
| | alkyl, aryl and their substituted derivatives^{a}, NH₂, HNR₅, | |
| | N(R₅)₂, (R₅ = C₁-C₁₂-alkyl, aryl and their substituted | |
| | derivatives^{a}); COR₆ (R₆ = H, NH₂, HNR₅, OR₅, C₁-C₁₂-alkyl, | |
| | aryl and their derivatives). R and R₁₋₆ each can be the same or different each time they appear in the formula. | |
| perhalomethyl triazines | 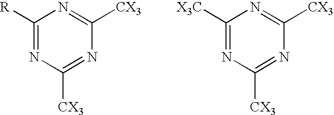 | HX |
| | X = halogen, R = methyl, phenyl, C, —C, 2-alkyl, aryl and their | |
| | substituted derivatives. | |
| Halobisphenyl A | 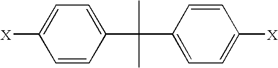 | HX |
| | X = halogen | |
| o-nitrobenzaldehyde | 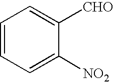 | 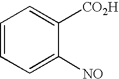 |
| sulfonates | 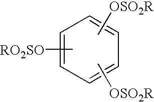 | RSO₃H |
| | R = CH₃, CF₃, Ph, C₁-C₁₂-alkyl, aryl | |
| | and their substituted derivatives. | |
| imidylsulfonyl esters | 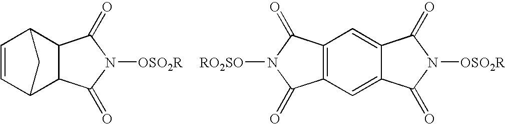 | RSO₃H |
| | 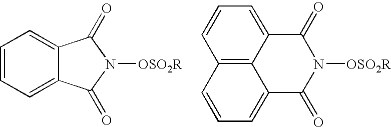 | |
| | R = CH₃, CF₃, Ph, C₁-C₁₂-alkyl, aryl | |
| | and their substituted derivatives. | |
| diaryliodonium salts | 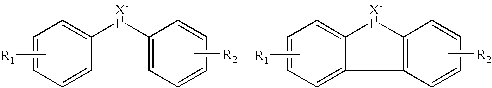 | HX₁BF₃ |
| | X = B(R₁)₄, AI(R₁)₄ (R₁ = halogen); R = H, halogen, NO₂, CN, | |
| | SO₂R₅, OH, OCH₃, SCH₃, CF₃, OR₅, SR₅, CH₃, t-butyl, C₁-C₁₂- | |
| | alkyl, aryl and their substituted derivatives^{a}, NH₂, HNR₅, | |
| | N(R₅)₂, (R₅ = C₁-C₁₂-alkyl, aryl and their substituted | |
| | derivatives^{a}); COR₆ (R₆ = H, NH₂, HNR₅, OR₅, C₁-C₁₂-alkyl, | |
| | aryl and their derivatives). R and R₁₋₆ each can be the same or | |
| | different each time they appear in the formula. | |
| sulfonium salts | 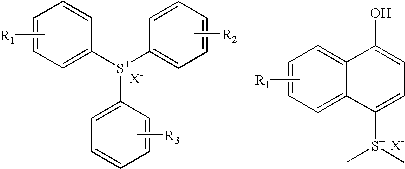 | HX₁BF₃ |
| | 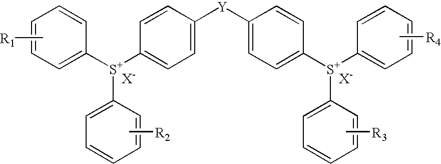 | |
| | 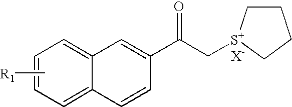 | |
| | X = B(R₁)₄, AI(R₁)₄ (R₁ = halogen); R = H, halogen, NO₂, CN, | |
| | SO₂R₅, OH, OCH₃, SCH₃, CF₃, OR₅, SR₅, CH₃, t-butyl, C₁-C₁₂- | |
| | alkyl, aryl and their substituted derivatives^{a}, NH₂, HNR₅, | |
| | N(R₅)₂, (R₅ = C₁-C₁₂-alkyl, aryl and their substituted | |
| | derivatives^{a}); COR₆ (R₆ = H, NH₂, HNR₅, OR₅, C₁-C₁₂-alkyl, | |
| | aryl and their derivatives). R and R₁₋₆ each can be the same or | |
| | different each time they appear in the formula. Y = O, S. | |
| diazosulfonate | 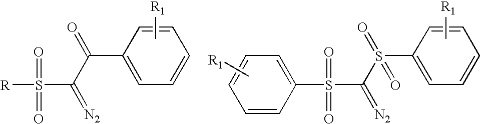 | RSO₃H R₁PhSO₃H |
| | R = phenyl, CH₃, CF₃, C₁-C₁₂-alkyl, aryl and their substituted | |
| | derivatives, R₁ = H, halogen, NO₂, CN, SO₂R₅, OH, OCH₃, | |
| | SCH₃, CF₃, OR₅, SR₅, CH₃, t-butyl, C₁-C₁₂-alkyl, aryl and their | |
| | substituted derivatives, NH₂, HNR₅, N(R₅)₂, (R₅ = C₁-C₁₂ alkyl, | |
| | aryl and their substituted derivatives); COR₆ (R₆ = H, NH₂, | |
| | HNR₅, OR₅, C₁-C₁₂-alkyl, aryl and their derivatives). R and R₁₋₆ | |
| | each can be the same or different each time they appear in the formula. | |
| diarylsulfones | 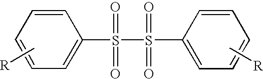 | 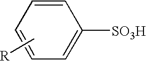 |
| | R = H, halogen, NO₂, CN, SO₂R₅, OH, OCH₃, SCH₃, CF₃, OR₅, | |
| | SR₅, CH₃, t-butyl, C₁-C₁₂-alkyl, aryl and their substituted | |
| | derivatives, NH₂, HNR₅, N(R₅)₂, (R₅ = C₁-C₁₂-alkyl, aryl and | |
| | their substituted derivatives); COR₆ (R₆ = H, NH₂, HNR₅, OR₅, | |
| | C₁-C₁₂-alkyl, aryl and their derivatives). R and R₁₋₆ each can be | |
| | the same or different each time they appear in the formula. | |
| 1,2-diazoketones | 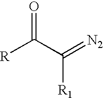 | 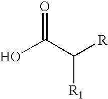 |
| | R, R₁ = H, halogen, NO₂, CN, SO₂R₅, OH, OCH₃, SCH₃, CF₃, | |
| | OR₅, SR₅, CH₃, t-butyl, C₁-C₁₂-alkyl, aryl and their substituted | |
| | derivatives^{a}, NH₂, HNR₅, N(R₅)₂, (R₅ = C₁-C₁₂-alkyl, aryl and | |
| | their substituted derivatives^{a}); COR₆ (R₆ = H, NH₂, HNR₅, OR₅, | |
| | C₁-C₁₂-alkyl, aryl and their derivatives). R and R₁₋₆ each can be | |
| | the same or different each time they appear in the formula. | |
| 2-diazo-1-oxo- 5-sulfonyls or 2-diazo-1-oxo-4- sulfonyl naphthanol esters | 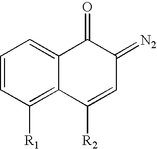 | 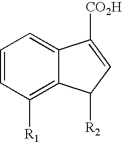 |
| | R₁, R₂ = H, SO₂R (R = C₁-C₁₂-alkyl, aryl, | |
| | and their substituted derivatives). | |
| | 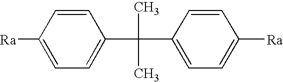 | |
| | 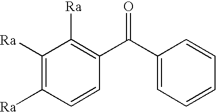 | |
| | 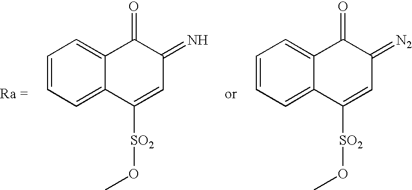 | |
| diazomethyl ketone | 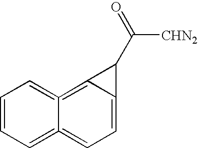 | 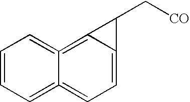 |
| diazo-Meldrums'acid | 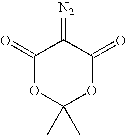 | 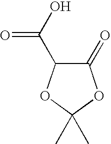 |
| arylazide derivatives | 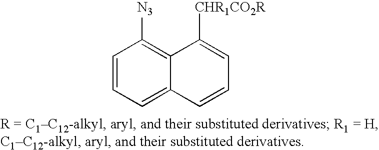 | 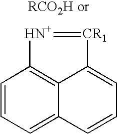 |
| arylazide derivatives | 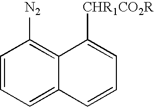 | HNR₂R₃ |
| | R = NR₂R₃, (R₂, R₃ = H, C₁-C₁₂-alkyl, aryl, and their | |
| | substituted derivatives), R1 = H, C₁-C₁₂-alkyl, aryl, and their substituted derivatives. | |
| benzocarbonates or carbamates | 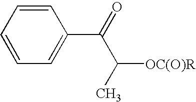 | RCO₂H or HNR₁R₂ |
| | R = NR₁R₂, (R₁, R₂ = H, C₁-C₁₂-alkyl, aryl, and their | |
| | substituted derivatives), C₁-C₁₂-alkyl, aryl, and their substituted derivatives. | |
| dimethoxybenzoinyl carbonates or carbamates | 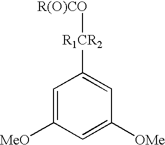 | RCO₂H or HNR₃R₄ |
| | R = NR₃R₄ (R₃, R₄ = H, C₁-C₁₂-alkyl, aryl and their substituted | |
| | derivatives), C₁-C₁₂-alkyl, aryl and their substituted derivatives; | |
| | R₁, R₂ = H, C₁-C₁₂-alkyl, COPh, aryl and their substituted derivatives. | |
| o-nitrobenzyloxy- carbonates or carbamates | 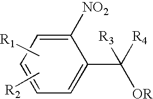 | R₅CO₂H R₅PO₃H R₅SO₃H or HNR₆R₇ |
| | R = COR₅ (R₅ = CF₃, OR₆, NH₂, HNR₆, C₁-C₁₂-alkyl, aryl and their or | |
| | derivatives (R₆ = H, C₁-C₃-alkyl, aryl and their substituted derivatives)), | |
| | SO₂R₅, PO₂R₅, CONR₆R₇ (R₇ = H, C₁-C₃-alkyl, aryl and their | |
| | substituted derivatives), R₁, R₂ = H, halogen, NO₂, CN, SO₂R₅, OH, OCH₃, ORₐ, | |
| | N(Rₐ)₂, (Rₐ = C₁-C₃ alkyl, aryl and their substituted derivatives); CH₃, t-butyl, | |
| | C₁-C₁₂-alkyl, aryl and their substituted derivatives; R₃, R₄ = H, | |
| | C₁-C₁₂-alkyl, aryl, and their substituted derivatives. | |
| nitrobenzene- sulphenyl | 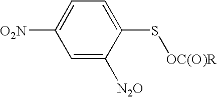 | RCO₂H or HNR₁R₂ |
| | R = CF₃, NR₁R₂ (R₁, R₂ = H, C₁-C₃-alkyl, aryl and their | |
| | substituted derivatives), C₁-C₁₂-alkyl, aryl and their derivatives. | |
| o-nitroanilines | 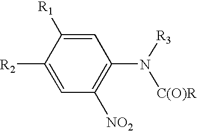 | RCO₂H or HNR₄R₅ |
| | R = CF₃, NR₄R₅ (R₄, R₅ = H, C₁-C₁₂-alkyl, aryl and their | |
| | substituted derivatives), alkyl, aryl and their derivatives; R₁, R₂ = | |
| | H, halogen, NO₂, CN, SO₂R₄, OH, OCH₃, ORₐ, N(R₄)₂; CH₃, | |
| | t-butyl, C₁-C₁₂-alkyl, aryl and their substituted derivatives; R₃ = | |
| | H, C₁-C₁₂-alkyl, aryl and their substituted derivatives. | |

References: Sus et al., Liebigs Ann. Chem. 556, 65-84 (1944); Hisashi Sugiyama et al., U.S. Pat. No. 5,158,885 (1997); Cameron et al., J. Am. Chem. Soc. 113, 4303-4313 (1991); Frechet, Pure & Appl. Chem. 64, 1239-1248 (1992); Patchornik et al., J. Am. Chem. Soc. 92, 6333-6335 (1970).

TABLE 1b
Examples of Radiation Sensitizers for PGR Reactions^{a}

| | | |
|---|---|---|
| photosensitizer | 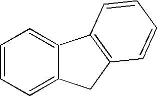 | 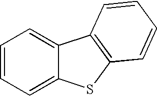 |
| | 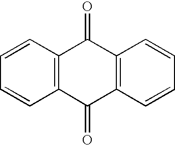 | 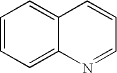 |

Photosensitizers include but not limited to the following:
benzophenone, acetophenone, benzoinyl C₁-C₁₂-alkyl
ethers, benzoyl triphenylphosphine oxide, anthracene,
thioxanthone, chlorothioxanthones, pyrene, Ru²⁺
complexes, their various substituted derivatives,
and the like.

TABLE 1C
Examples of Stabilizers for PGR Reactions^{a}

| | | | | |
|---|---|---|---|---|
| R—H stabilizer | 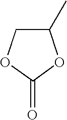 | 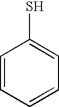 | 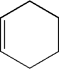 | 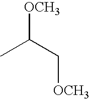 |

R—H stabilizers include but not limited to the following:
propylene carbonate, propylene glycol ethers, t-butane,
t-butanol, thiols, cyclohexene, their substituted derivatives
and the like.
^{a}Substituted derivatives contain at least one of the substituent groups, which include but not limited to halogen, NO₂, CN, OH, SH, CF₃, C(O)H, C(O)CH₃, C₁-C₃-acyl, SO₂CH₃, C₁-C₃—SO₂R₂, OCH₃, SCH₃, C₁-C₃—OR₂,
# C₁-C₃—SR₂, NH₂, C₁-C₃—NHR₂, C₁-C₃, —N(R₂)₂ (R₂ = alkyl, can be the same or different each time they appear in the formula).

One embodiment of the present invention contemplates PGRPs used in combination with co-reagents, such as radiation sensitizers. Many radiation sensitizers are known to those skilled in the art and include those previously mentioned. It is to be understood that one of ordinary skills in the art will be able to readily identify additional radiation sensitizers based upon the present disclosure. Preferably, a radiation sensitizer is a photosensitizer has a lower excitation energy than the PGRP used. In a preferred embodiment, illumination excites a photosensitizer, which in turn initiates the conversion of a PGRP to give a PGR. Specifically, a photosensitizer shifts the excitation wavelength used in photochemical reactions and enhances the efficiency of the formation of PGRs.

In one embodiment of the present invention, the substrate surface is solid and substantially flat. As nonlimiting examples, the substrate can be a type of silicate, such as glass, Pyrex or quartz, a type of polymeric material, such as polypropylene or polyethylene, and the like. The substrate surfaces are capable of fabrication and derivatization consistent with the various embodiments of the present invention contemplated herein.

Photo-Generated Acid Deprotection and Oligonucleotide Synthesis

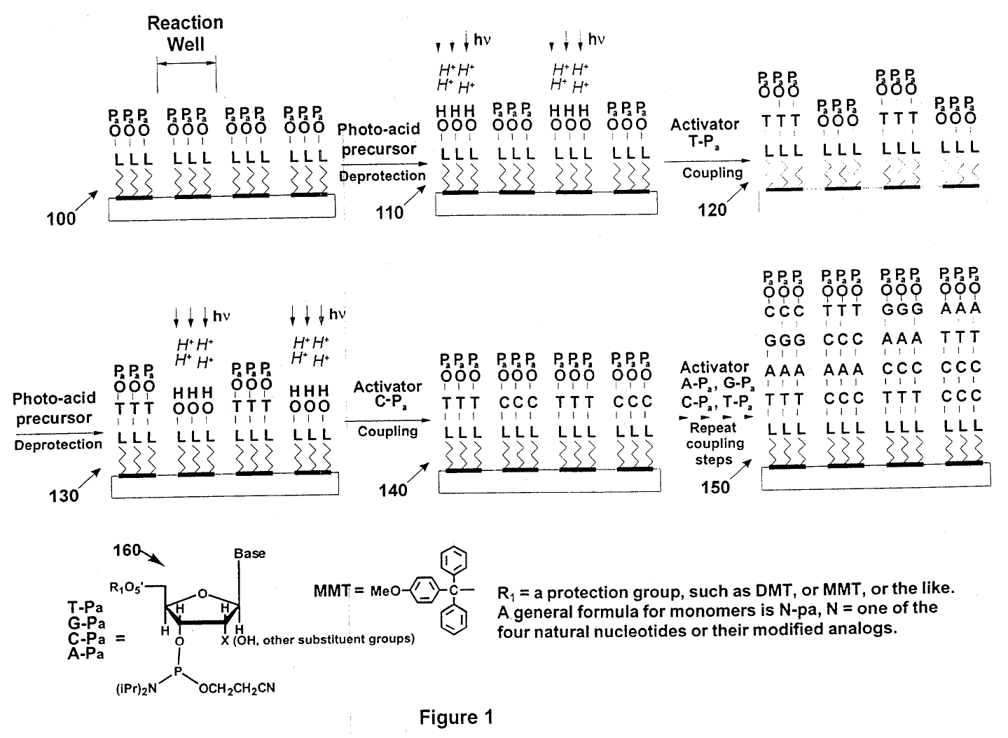

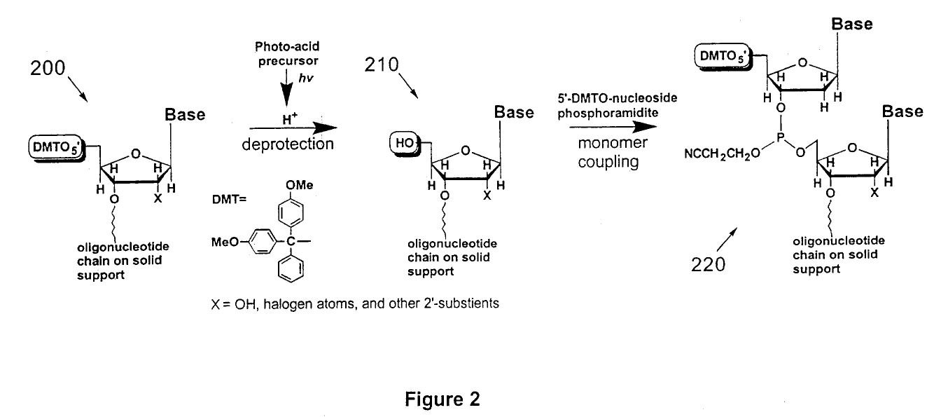

One embodiment of the present invention contemplates a linker (i.e., an “initiation moiety” that also broadly includes monomers or oligomers on which another monomer can be added) that is attached to a solid substrate surface and provides an anchor on which oligonucleotide biopolymer sequences are synthesized. (see FIGS. 1 and 2) Preferably, the methods contemplated by the present invention for synthesis of oligonucleotides are based on known protocols. McBride et al., Tetrahedron Letter 24, 245-248 (1983) For example, as shown in FIG. 1, each linker molecule contains a reactive terminal group, such as 5′-OH, protected by an acid-labile protecting group, as shown in Step 100. Next, a PGRP either with, or without a photosensitizer are applied to a solid substrate. Step 110 illustrates the projection of a controlled light pattern onto the solid substrate. In the presence of a mixture comprising an acid PGRP, acid PGRs are produced at illuminated isolated reaction sites, causing cleavage of acid-labile protecting group (such as DMT) from the 5′-OH, and the terminal-OH groups are free to react with incoming monomers. (see FIG. 2) On the other hand, no acid is produced at non-illuminated isolated reaction sites and, therefore, the acid-labile protecting groups on these linker molecules are not cleaved. The solid substrate comprising the deprotected linker is then washed and subsequently contacted with a first nucleotide monomer (e.g., for example, a nucleophosphoramidite, a nucleophosphonate or an analog compound). Step 120 demonstrates the coupling of the first nucleotide monomer to deprotected linkers using conventional reaction conditions. A bond, either covalent or non-covalent, is thus formed between the OH groups of the linker and an unprotected reactive terminal group, (i.e., for example, phosphophate, amine, sulfydryl, etc.) of the first monomer. After proper washing, oxidation and capping steps, the addition of the first nucleotide monomer is complete.

The bonding of the linker and a first, or subsequent, nucleotide monomer creates a multimer that comprises a reactive terminal group (i.e, for example, an OH group) and is protected by an acid-labile group. The solid substrate attached to the multimers is then contacted with a second acid PGRP and a second controlled light pattern (see Step 130). The illuminated isolated reaction sites containing multimers comprising acid-labile protecting groups are deprotected by the conversion of the acid PGRP into acid PGRs. This is followed by washing and contacting the solid substrate with a second nucleotide monomer. The second nucleotide monomer forms a bond, either covalent or non-covalent, with the multimer only at deprotected reactive terminal groups. The second nucleotide monomer also comprises a reactive terminal group protected by an acid-labile group (see Step 140). Finally, as indicated in Step 150, this addition of subsequent nucleotide monomers to the multimer is repeated until at least one oligonucleotide of the desired length and nucleotide sequence is formed at at least one isolated reaction site.

For a microarray or biochip containing an oligonucleotide of a particular designated sequence, the maximum number of reaction steps is 4×n, where n is the chain length and 4 is a constant for natural nucleotides. Biopolymers containing modified sequences may require more than 4×n steps.

While the above describes a series of steps, one of ordinary skill in the art would recognize that somes steps are, or may be, performed simultaneously. For example, the protected monomers can be in the mixture at the time of illumination.

Photo-Generated Activator Mediated Coupling Reaction and Oligonucleotide Synthesis

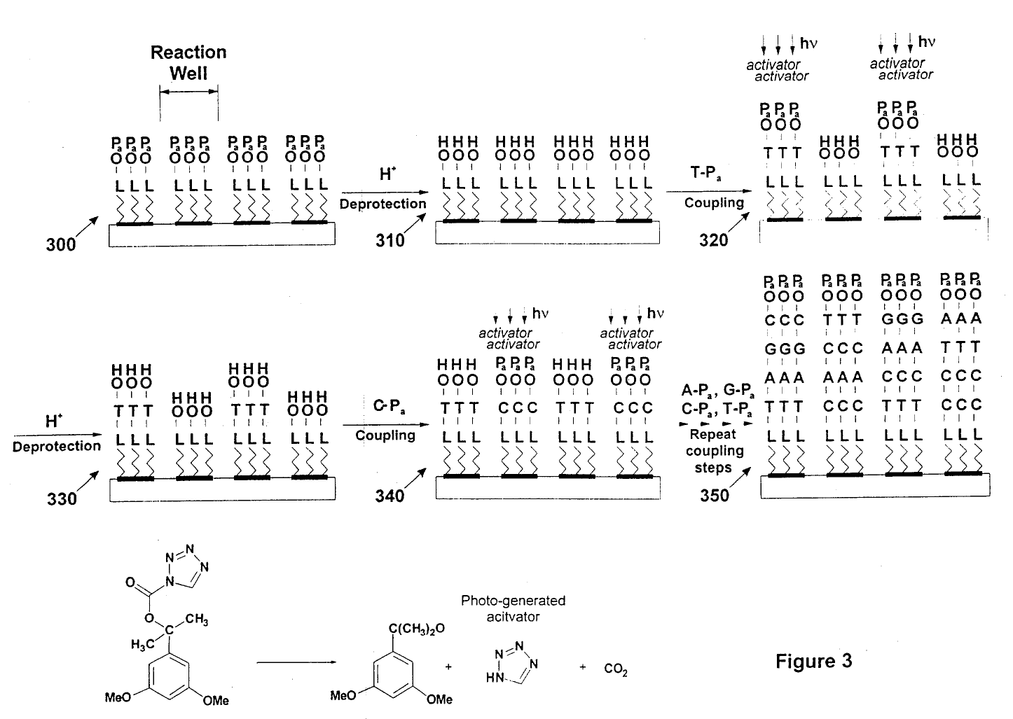

One embodiment of the present invention contemplates a photo-generated activator precursor comprising a first nucleotide monomer (PGAP; for example a compound containing tetrazole linked to a photolabile group). (see FIG. 3) Preferably, as shown in Step 300, linkers are attached to a solid substrate on which oligonucleotide sequences are to be synthesized. Next, in Step 310 acid-labile protecting groups on linkers are deprotected by a conventional acid. Next, a mixture comprising a PGAP either with, or without, a photosensitizer is contacted with the solid substrate. As shown in Step 320, a controlled light pattern is then projected onto the solid substrate. At illuminated isolated reaction sites, PGAPs are converted into an activated state that faciliates coupling of the first nucleotide monomer to the linker. At non-illuminated isolated reaction sites PGAPs are not converted into an activated state and, therefore, no first nucleotide monomer-linker coupling occurs. After proper washing, oxidation and capping steps, the addition of the first monomer is complete.

The bonding of the attached first nucleotide monomer to the linker creates a multimer that also contains a protected reactive terminal group. The solid substrate attached to the multimers is then contacted with a second conventional acid. (see Step 330) The second conventional acid deprotects reactive terminal groups on the multimer. Subsequently, the solid substrate is washed and contacted with the second nucleotide monomer. As demonstrated in Step 340, the second nucleotide monomer couples only at deprotected reactive terminal groups. Finally, as indicated in Step 350, the elongation of the multimer is repeated until at least one oligonucleotide of a desired length and chemical sequence is formed at at least one isolated reaction site.

Alternative Embodiments of Oligonucleotide Synthesis using Photo-Generated Reagents

Some alternative embodiments of the present invention contemplate the use of nucletide analogs as the nucleotide monomer exemplified by coupling Steps 120, 140, 150, 320, 340 and 350. The synthesis of oligonucleotides using nucleotide analogs proceeds as described in FIGS. 1 and 3 to generate oligonucleotides containing modified residues.

Other alternative embodiments of the present invention contemplate an acid-labile protecting group, such as DMT, at the 3′-OH position of the nucleotide monomer exemplified by coupling Steps 120, 140, 150, 320, 340 and 350. The synthesis of these reverse-oriented oligonucleotides using 3′-OH protected nucleotide monomers proceeds as described in FIGS. 1 and 3 to generate oligonucleotides having a sequence oriented in an opposite orientation as compared to those using 5′-OH protected monomers. Such reverse-oriented oligonucleotides are of particular use as primers for in situ polymerase chain reactions (i.e., PCR).

Photosensitizers

The use of PGR in the present invention permits chemical/biochemical reactions under conventional conditions. In one embodiment, the occurrence of the synthesis reaction is controlled by in situ formation of at least one reagent upon illumination (i.e., for example, light). Preferably, such illumination is from a light source emitting ultraviolet (UV) and visible light but heat, infrared (IR) and X-ray illumination are also contemplated sources of illumination.

A PGR may be produced by illumination of a PGRP or a photosensitizer. Specifically, a photosensitizer transfers energy to a PGRP. Subsequent to the energy transfer, a chemical transformation occurs to yield at least one product (i.e., a PGR), which is a stable intermediate compound. The PGR is formed by a portion of the PGRP that is dissociated from the parent, or a rearranged, structure of the PGRP. The PGR may be selected from the group consisting of an acid, a base, a nucleophile, an electrophile, or other reagents having specific reactivities in accordance with the properties of the compounds specified in Table 1.

One embodiment of the present invention contemplates an improved reaction yield and/or suppression of side reactions achieved by illumination of at least one PGRP prior to contacting the PGRP with the isolated reaction sites. This prior illumination of PGRPs allows time for unstable reaction intermediates, such as free radical species generated during illumination, to diminish and for stable PGRs, such as H⁺, to reach a steady-state concentration. Preferably, a combination with at least one stabilizer improves reaction yield and/or suppresses side reactions.

One example of a photosensitizer contemplates a reduced lifetime of unstable reaction intermediates, such as a free radical species generated during illumination, and the production of a low energy source comprising hydrogen. This example is illustrated by the following reactions that generate H⁺ from sulfonium salts (Ar₃S⁺X⁻):

$$[ {Ar}_{3} {S}^{+} ] {X}^{-} {⟶}^{hv} {Ar}_{2} {S}^{+ *} + {X}^{-} + {Ar}^{*} {⟶}^{RH} {Ar}_{2} {SH}^{+} + {X}^{-} + RAr ⟶ {Ar}_{2} S + {H}^{+} {X}^{-} + RAr$$

Specifically, the RH compounds in the above equation are stable and are good H⁺ donors. Other, non-limiting examples of such photosensitizer compounds include propylenecarbonate (one of the major components of UVI 6974 and UVI 6990), t-butane, cyclohexene, and the like (See Table IC).

Photosensitizers within the scope of the present invention include any compound that is sensitive to illumination and able to improve excitation profile of a PGR by shifting its excitation wavelength and enhancing illumination efficiency. Examples of photosensitizers include, but are not limited to, benzophenone, anthracene, thioxanthone, their derivatives (Table 1B), and the like.

Photo-Generated Reagents

PGRPs within the scope of the present invention include any compound that produces an acid PGR upon illumination (i.e. for example, light). Examples of PGRPs generating acid PGRs include, but are not limited to, diazoketones, triarylsulfonium, iodonium salts, o-nitrobenzyloxycarbonate compounds, triazine derivatives, and the like. (see Table 1A)

In one embodiment, a PGRP is a triarylsulfonium hexafluoroantimonate derivative and generates an acid PGR (Dektar et al., J. Org. Chem. 53, 1835-1837 (1988); Welsh et al., J. Org. Chem. 57, 4179-4184 (1992); DeVoe et al., Advances in Photochemistry 17, 313-355 (1992)). This group of compounds belong to a family of onium salts, which undergo photogenerated reactions, either directly or in the presence of a photosensitizer, to form free radical species and finally generating diarylsulfides and H⁺ (supra).

In another embodiment, a PGRP is diazonaphthoquionesulfonate triester ester, which produces indenecarboxylic acid upon UV illumination at λ>350 nm. The formation of this acid is due to a Wolff rearrangement through a carbene species to form a ketene intermediate and the subsequent hydration of ketene (Sus et al., Liebigs Ann. Chem. 556, 6584 (1944); Sugiyama et al., U.S. Pat. No. 5,158,885 (1997)).

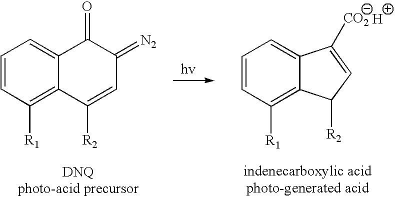

These intermediates and products produced by PGRPs have been extensively used in cationic and radical catalyzed polymerizations for high-resolution microimaging photolithograpy. Specifically, acid-producing PGRPs have been widely used for many years in printing and microelectronics industries as a component in photoresist formulations. Willson, In “Introduction To Microlithography”, Thompson et al. Eds., Am. Chem. Soc.: Washington D.C., (1994) These reactions are, in general, fast (complete in a matter of seconds or minutes), proceed under mild conditions (room temperature, neutral solution), and the solvents used in the photoreactions (haloalkanes, ketones, esters, ethers, toluene, and other protic or aprotic polar solvents) are compatible with oligonucleotides or other organic chemical reactions. McBride et al., Tetrahedron Letters, 24, 245-248 (1983) One embodiment of the present invention contemplates selecting an acid PGRP listed in Table 1 that minimize side reactions in the desired synthesis protocol. Specifically, PGRP chemical properties, such as acidity of the acid PGRs, can be adjusted by placing different substitution groups on the ring or chain moieties. In one specific embodiment, the electronegative sulfonate group in the indenecarboxylic acid stabilizes the negative charge on the carboxylic group attached to the same ring moiety to produce a PGR that effectively deprotects the 5′-O-DMT group in a way comparable to that of using the conventional trichloroacetic acid (TCA). (see FIG. 2) In general, electron-withdrawing groups, such as —O₂SOR, —NO₂, halogens, —C(═O)R(R=aryl, alkyl, and their substituted derivatives, or —XR, (X═S, O, N; R1=aryl, alkyl, and their substituted derivatives) increase the strength of the corresponding acids (i.e., PGRs). On the other hand, electron donating groups, such as —OR (R=aryl, alkyl, and their substituted derivatives) decrease the acidity of the PGRs. The ability to generate acid PRGs having widely different pKa's makes the present invention applicable to most any acid-catalyzed deprotection reaction.

PGRPs within the scope of the present invention include any compound that produce basic PGRs upon illumination. Examples of such PGRPs that convert into basic PGRs include, but are not limited to, o-benzocarbamates, benzoinylcarbamates, nitrobenzyloxyamine derivatives and the like. (see Table 1) In a preferred embodiment, the present invention contemplates PGRPs containing amino groups protected by photolabile groups that release amines in quantitative yields upon contacting illumination (i.e., for example, light). These PGRPs are then converted into amine-containing PGRs that create a relatively basic (i.e., a higher pH) environment.

Alternative PGRPs are also within the scope of the present invention that include any compound which, subsequent to illumination, produces a PGR required for a chemical/biochemical reaction. In a preferred embodiment, the present invention contemplates compounds including, but not limited to, 1-(dimethoxylbenzoinyl)tetrazole (heterocyclic compound tetrazole is a PGR), dimethoxylbenzoinylOR, (R₁OH is a PGR, R₁=alkyl, aryl and their substituted derivatives), sulfonium salts (thiol ether Ar₂S is a PGR), and the like.

Synthesis of Polypeptides using PGRs

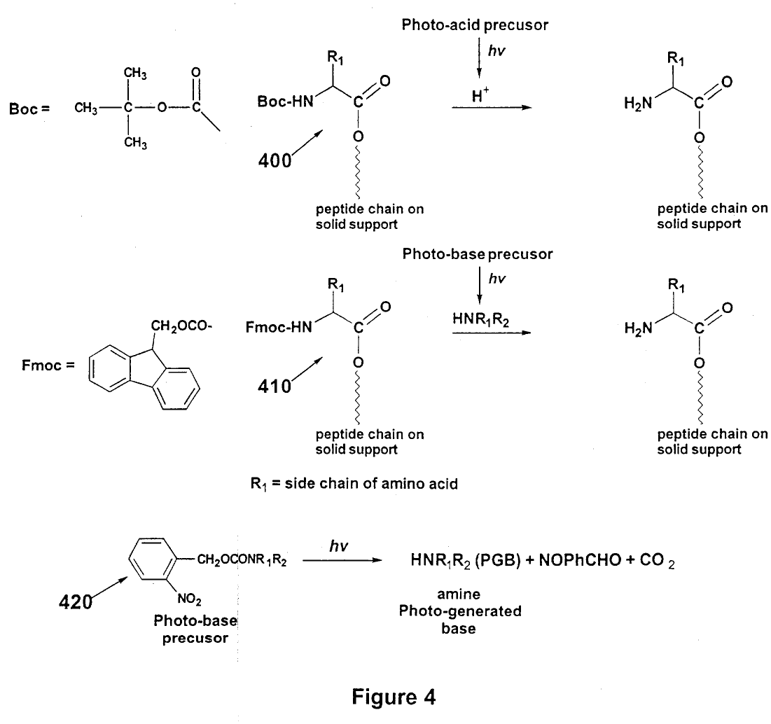

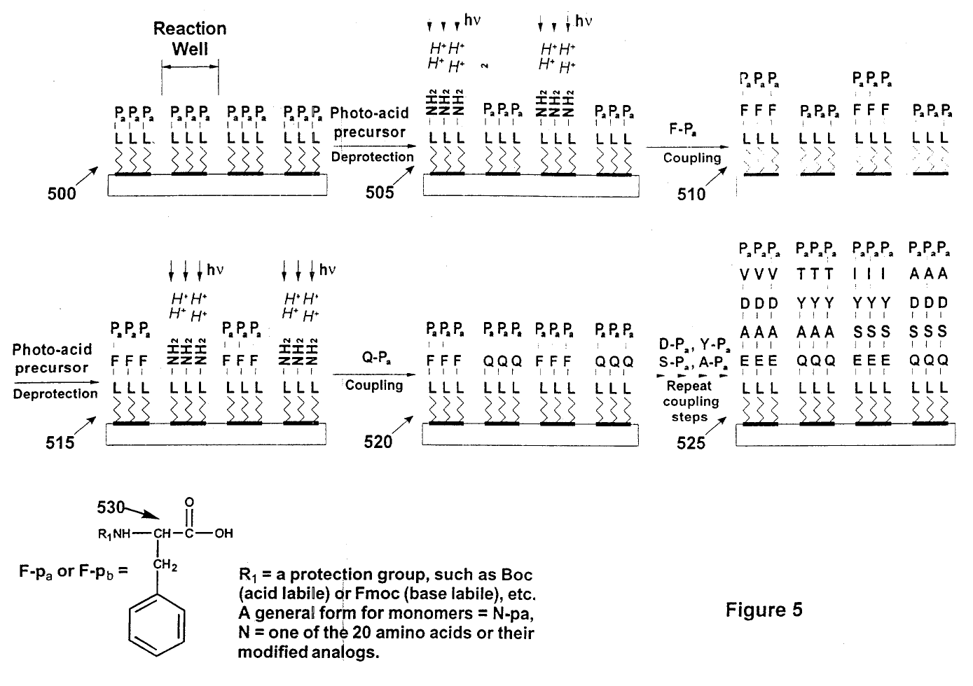

One embodiment of the present invention contemplates PGRs that are applied to solid substrates (i.e., for example, microarrays or biochips) to facilitate the parallel synthesis of multiple peptide sequences using amino acid monomers containing reactive terminal groups protected by t-Boc (acid-labile) or Fmoc (base-labile) groups (see FIG. 4). The methods of present invention contemplate using PGR-mediated peptide synthesis by conventional chemistry. Sterwart and Young, “Solid phase peptide synthesis”, Pierce Chemical Co.; Rockford, Ill. (1984); Merrifield, Science 232, 341 347 (1986); and Pirrung et al., U.S. Pat. No. 5,143,854 (1992).

One embodiment of the present invention contemplates a linker attached to a solid substrate on which a peptide sequence is synthesized. Preferably, each linker contains a reactive terminal group, such as an —NH₂ group, protected by the acid labile t-Boc group. (see Step 500) Next, as illustrated in Step 505, a mixture comprising an acid PGRP either with, or without a photosensitizer, is contacted with a solid substrate and a controlled light pattern is then projected onto the solid substrate. At illuminated isolated reaction sites, acid PGRs are produced thus cleaving the acid-labile protecting groups (i.e., for example, t-Boc) from the N-terminal —NH₂. At non-illuminated isolated reaction sites, no acid is produced and, therefore, no cleavage of the acid-labile protecting groups occurs. The solid substrate is subsequently washed and contacted with a first amino acid monomer (i.e., for example, a protected amino acid, its analogs, or oligomers), which bonds, either covalently or non-covalently, only to a deprotected linker under conventional coupling reaction conditions (see Step 510). Preferably, a covalent bond is formed between the —NH₂ group of a linker and a carbonyl carbon of a first amino acid monomer to create an amide linkage. After proper washing steps, the addition of the first amino acid monomer is complete, thus forming a multimer. In a preferred embodiment, said first amino acid monomer comprises a reactive terminal group protected by the acid-labile t-Boc group.

Next, as illustrated in Step 515, a solid substrate comprising a multimer is a second acid PGRP and exposed to a second controlled light pattern. The protected multimers attached at illuminated isolated reaction sites are deprotected following the conversion of a second acid PGRP into a second acid PGR. Following washing, a second amino acid monomer is contacted with a deprotected multimer and forms a bond, either covalently or non-covalently. Preferably, said second amino acid monomer contains a reactive terminal group protected by an acid-labile group (see Step 520). Finally, as indicated in Step 525, the elongation of the multimer is repeated until a polypeptide of a desired length and sequences is formed at at least one isolated reaction site. For a microarray or biochip containing a polypeptide having a designated sequence, the maximum number of reaction steps is 20×n, where n is the chain length and 20 is the number of naturally occurring amino acids. The synthesis of polypeptide sequences containing modified amino acids may require more than 20×n steps.

While the above describes a series of steps, one of ordinary skill in the art would recognize that somes steps are, or may be, performed simultaneously. For example, the protected monomers can be in the mixture at the time of illumination.

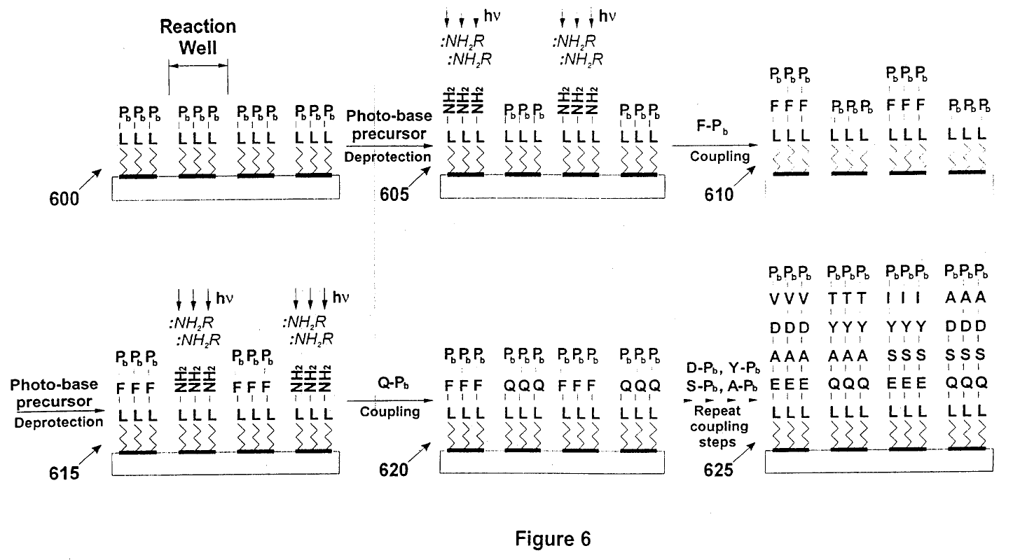

Another embodiment of the present invention contemplates polypeptide synthesis using a basic PGRP (see FIG. 6). Preferably, as depicted in Step 600, a solid surface comprises a linker that contains a reactive terminal group, such as —NH₂, protected by a base-labile group. Next, a mixture comprising a base PGRP (i.e., for example, (((2-nitrobenzyl)oxy)carbonyl)piperidine; Cameron and Frechet, J. Am. Chem. Soc. 113, 43034313 (1991)) is applied to a solid substrate and a controlled light pattern is projected onto said solid substrate (see Step 605). At illuminated isolated reaction sites, base PGRs are produced cleaving base-labile protecting groups from the linkers, thus creating deprotected linkers. At non-illuminated isolated reaction sites, no base PGR is produced and the base-labile protecting groups are not cleaved from said linkers. Next, Step 610 shows that the solid substrate is subsequently washed and contacted with a first amino acid monomer containing a carboxylic acid reactive terminal group which forms a bond, either covalently or non-covalently, only to said deprotected linkers according to conventional chemistry. Preferably, after proper washing, the addition of the first amino acid monomer is completed thus forming a multimer by the creation of an amide linkage. In a preferred embodiment, said second amino acid monomer comprises a reactive terminal group protected by a base-labile group.

Next, as depicted in Step 615, a solid substrate containing said multimers is contacted with a mixture comprising a second base PGRP and exposed to a second controlled light pattern. Multimers attached to illuminated isolated reaction sites are deprotected due to the conversion of said second base PGRP into a second base PGR. Subsequently, the solid substrate is washed and contacted with a second amino acid monomer. As shown in Step 620, said second amino acid monomer forms a bond, either covalently or non-covalently only to multimers attached to illuminated isolated reaction sites. Peferably, said second amino acid monomer bonded to said multimer comprises a reactive terminal group protected by a base-labile group. This multimer elongation process is repeated until a polypeptide of a desired length and amino acid sequence is formed at at least one isolated reaction site.

While the above describes a series of steps, one of ordinary skill in the art would recognize that somes steps are, or may be, performed simultaneously. For example, the protected monomers can be in the mixture at the time of illumination.

The Synthesis of Oligosaccharides using PGRs

The present invention is not limited to the parallel synthesis of biopolymers comprising oligonucleotides and polypeptides. The method is of general use in solid phase synthesis of molecular arrays where complex synthesis patterns are required at each step of chain extension synthesis.

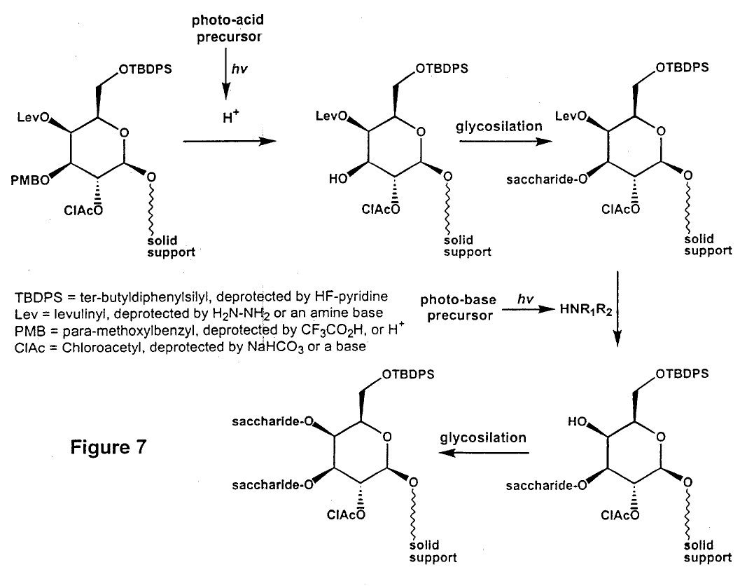

One embodiment of the present invention contemplates synthesis of oligosaccharide sequences comprising diverse saccharide monomers and branched chains (see FIG. 7). According to a preferred embodiment of the present invention, a mixture comprising an acid PGRP is contacted with a solid surface attached to a protected saccharide monomer. Preferably, each saccharide monomer contains several reactive OH groups, each of which is bonded to a protecting group and each requiring different deprotection conditions. Next, a controlled light pattern is then projected onto the solid substrate, wherein at illuminated isolated reaction sites, an acid PGR is produced and a first protected OH group is cleaved thus forming a first deprotected OH group. At non-illuminated isolated reaction sites, no acid PGR is produced and, therefore, acid-labile protecting groups bonded to said first protected OH group is not cleaved. Next, the solid substrate is washed and contacted with a first saccharide monomer, wherein said first saccharide monomer may consist of a carbohydrate or an oligosacchride. In one embodiment, said first saccharide monomer contains acid-labile protecting groups at all but one OH groups. Preferably, said first saccharide monomer bonds, either covalently or non-covalently, only to said first deprotected OH group according to conventional chemistry that creates a glycosidic linkage, thus forming a multimer. Wong et al., J. Am. Chem. Soc. 120, 7137-7138 (1998). The multimer elongation process is repeated by adding subsequent saccharide monomers in the presence of acid PGRs to synthesize an oligosacchride containing various glycosidic linkages at said first deprotected OH group.

Another embodiment of the present invention contemplates continuing the above described, or initiating a new, polysaccharide synthesis using a base PGRP by contacting said base PGRP with a solid substrate attached to a saccharide monomer, wherein said monomer may consist of a carbohydrate or an oligosaccharide, comprising protected OH groups having differential susceptibility to cleavage. Next, a second controlled light pattern is then projected onto the solid substrate wherein at illuminated isolated reaction sites a base PGR is converted from said base PGRP and a protecting group on a second OH group is cleaved, thus creating a second deprotected OH group. At non-illuminated isolated reaction sites, no base PGR is produced from said base PGRP and, therefore, the base-labile protecting groups of said protected second OH group is not cleaved. Subsequently, the solid substrate is washed and contacted with a second saccharide monomer, which bonds, either covalently or non-covalently only to said second deprotected OH group according to conventional chemistry to form a glycosidic linkage, thereby creating a multimer. Preferably, said second saccharide monomer comprises base-labile protecting groups at all but one OH group. The multimer elongation process is repeated to synthesize an oligosacchride containing various glycosidic linkages at said second deprotected OH position.

Another embodiment of the present invention contemplates alternation of acid PGR and base PGR mediated saccharide monomer synthesis such that branched oligosacchrides are formed. Preferably, when continued synthesis is contemplated various PGRs are used to achieve selective deprotection of the OH protecting groups until at least one desired oligosacchride sequence is synthesized.

The present invention contemplates the use of PGRPs in other applications besides deprotection reactions. Specifically, photo-generated reactive compounds, such as alcohols (ROH, R=alkyl, aryl and their substituted derivatives), are contemplated as reagents for a variety of chemical conversions, such as esterification, nucleophilic substitution and elimination reactions. These reactions are relevant to the fabrication of custom microarrays and biochips (infra).

II. The Synthesis Apparatus

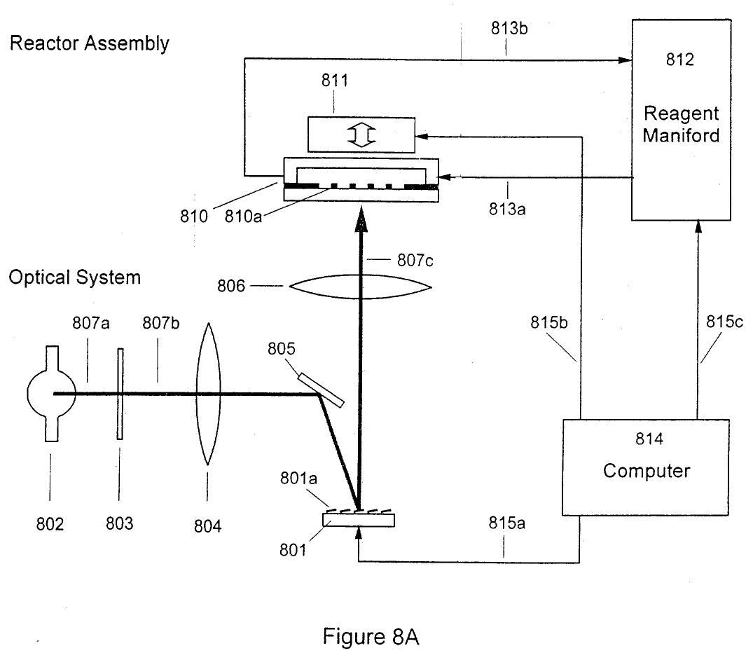

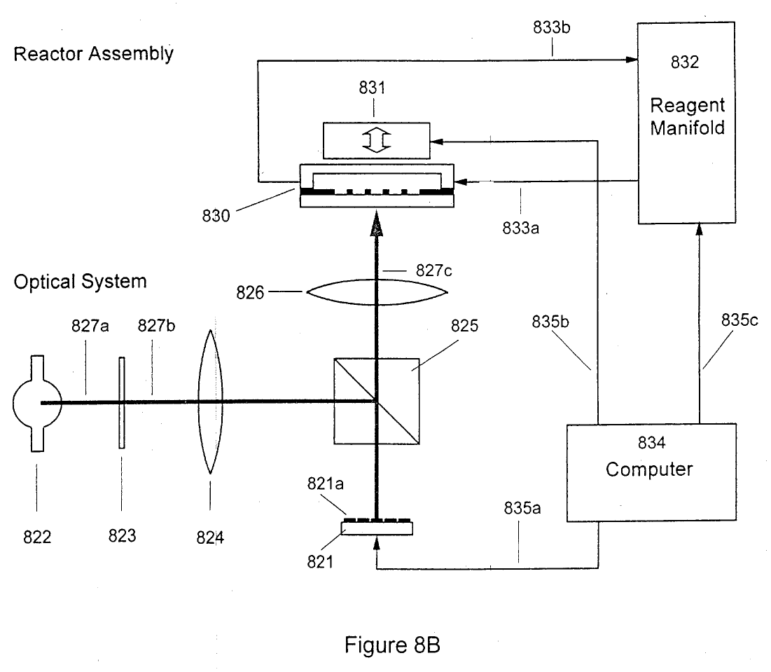

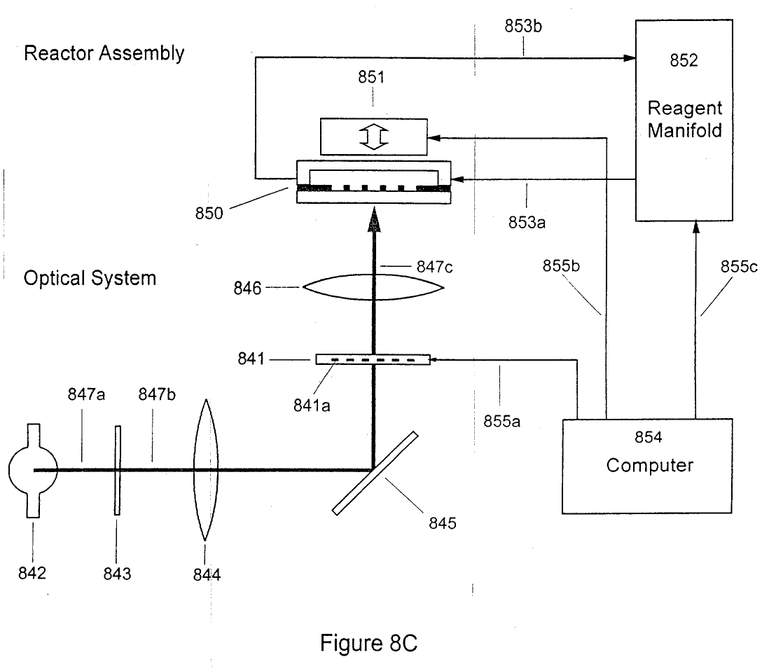

FIGS. 8A thru 8C illustrate three embodiments of the programmable, light-directed synthesis apparatus of this invention. As shown in FIG. 8A, the apparatus is comprised of four sections: a reagent manifold 812, an optical system, a reactor assembly, and a computer 814, each of which is described further below.

Reagent Manifold

The reagent manifold 812 of FIG. 8A performs standard reagent metering, delivery, circulation, and disposal. It consists of reagent containers, solenoid or pneumatic valves, metering valves, tubing, and process controllers (not shown in FIG. 8A). The reagent manifold 812 also includes an inert gas handling system for solvent/solution transport and line purge. The design and construction of such a manifold are well known to those who are skilled in the art of fluid and/or gas handling. In many cases, commercial DNA/RNA, peptide, and other types of synthesizers can be used as the reagent manifold 812 of this invention.

Optical System

The function of the optical system shown in FIG. 8A is to produce patterned light beams or controlled light patterns 807c for initiating photochemical reactions at isolated reaction-wells on a solid substrate 810a. The optical system shown in FIG. 8A is comprised of a light source 802, one or more filters 803, one or more condenser lenses 804, a reflector 805, a Digital Micromirror Device (DMD) 801, and a projection lens 806. During operation, a light beam 807a is generated by the light source 802, passes through the filter(s) 803, and becomes a light beam 807b of desired wavelength. A condenser lens 804 and a reflector 805 are used to direct the light beam 807b on to the DMD 801. Through a projection lens 806, the DMD projects a light pattern 807c on the solid substrate 810a of a reactor assembly 810. Details about the DMD 801 are described below.

A light source 802 may be selected from a wide range of light-emitting devices, such as a mercury lamp, a xenon lamp, a halogen lamp, a laser, a light emitting diode, or any other appropriate light emitter. The wavelengths of the light source 802 should cover or fall within the excitation wavelengths of the desired photochemical reaction. The preferred wavelengths for most photochemical reactions relevant to embodiments contemplated by the present invention are between 280 nm and 500 run. The power of the light source 802 should be sufficient to generate a light pattern 807c intense enough to complete the desired photochemical reactions in a reactor assembly 810 within a reasonable time period. For most applications, the preferred light intensity contacting the solid substrate 810a is between 0.1 to 100 mW/cm². For many applications, a mercury lamp is preferred due to its broad wavelengths and availability of various powers.

Selection criterions for a filter(s) 803 are based on the excitation wavelength of the desired photochemical reactions and other considerations. In one embodiment of the present invention, it is contemplated to remove undesirably short and long wavelengths from the light beam 807a in order to avoid unwanted photodegradation reactions and heating in a reactor assembly 810. For example, in the synthesis of oligonucleotides and other biopolymers, it is preferred to remove wavelengths shorter than 340 nm. To avoid heating, an infrared cut-off filter is preferably used to remove wavelengths beyond 700 nm. In a preferred embodiment, more than one filter is contemplated.

The Optical System shown in FIG. 8A contemplates a Digital Micromirror Device 801, which is used to generate light patterns 807b. A DMD is an example of an electronically controlled optical modulator device that is capable of producing graphical and text images in the same manner as a computer monitor. The DMD is commercially available from Texas Instruments Inc., Dallas, Tex. USA, for projection display applications. Hornbeck, L. J., “Digital light processing and MEMS, reflecting the digital display needs of the networked society,” SPIE Europe Proceedings, 2783, 135-145 (1996) Each DMD 801 contains a plurality of small and individually controllable rocking-mirrors 801a, which steer light beams to produce images or light patterns 807c. DMD 801 is a preferred means of producing controlled light patterns in the present invention for several reasons. First, the DMD 801 is capable of handling relatively short wavelengths that are needed for initiating desired photochemical reactions. Second, the DMD 801 has high optical efficiency. Third, the DMD 801 can produce light patterns having a high contrast ratio. In addition, versions of DMD 801 having high resolution formats (up to 1920×1080) have been demonstrated. These features permit one to conveniently generate optical patterns for the synthesis of practically any desired biopolymer sequence by using the photochemistry contemplated in various embodiments of this invention. In this aspect, the synthesis apparatus contemplated by this invention is highly flexible as compared with previous methods of synthesizing biopolymers using photomasks.

Many types of electronically controlled optical modulator devices are contemplated by this invention for generating controlled light patterns. FIG. 8B illustrates an exemplary embodiment of the present invention, using a reflective liquid crystal array display (LCD) device 821. Reflective LCD devices are commercially available from a number of companies, such Displaytech, Inc. Longmont, Colo. USA. Each reflective LCD device 821 contains a plurality of small reflectors (not shown) with a liquid crystal shutter 821a placed in front of each reflector that controls the production of images or light patterns. High-resolution devices, up to 1280×1024 pixels, are already available from Displaytech. The optical system shown in FIG. 8B is similar to the device of FIG. 8A except for the optical arrangement for directing light onto optical modulator devices. A beam splitter 825 is used in the optical system shown in FIG. 8B to effectively guide light onto and out of the reflectors.

Another embodiment of the present invention contemplates a transmissive LCD display 841 to generate controlled light patterns, as shown in FIG. 8C. A transmissive LCD display 841 contains a plurality of liquid crystal light valves 841a, shown as short bars in FIG. 8C. When a liquid crystal light valve 841a is on, light passes; when a liquid crystal light valve is off, light is blocked. A transmissive LCD display, therefore, can be used in the same way as a conventional photomask is used in a standard photolithography process. Thompson et al., Introduction to Microlithography, American Chemical Society, Washington, D.C. (1994) In FIG. 8C, a reflector 845 is shown directing a light beam 847b to the transmissive LCD display 841. Most commercially available optical modulator devices, including DMD, reflective LCD, and transmissive LCD are designed for handling visible light (400 nm to 700 nm). The use of the instrument and the methods contemplated by this invention, however, extends beyond the above wavelength range.

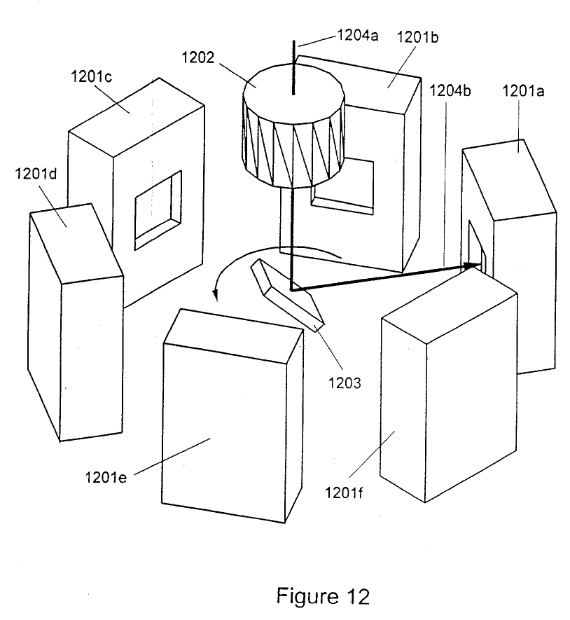

FIGS. 8A through 8C depict apparatus designs for making one microarray or biochip at a time, however, one embodiment of the present invention simultaneously produces a plurality of microarrays or biochips. FIG. 12 presents one embodiment of a stepping mechanism that improves bipolymer synthesis throughput and the efficiency. In this stepping mechanism, a light beam 1204a is projected from an optical modulator device, (not shown in the figure) passes through a projection lens 1202, and is directed by a reflector 1203 towards a reactor assembly 1201a forming an image or a controlled light pattern 1204b. The reflector 1203 is capable of rotation that can direct said controlled light pattern 1204b towards any one of several surrounding reactor assemblies (i.e., for example, 1201a through 1201f). In a regular synthesis process of, for example, oligonucleotides, the controlled light pattern 1204b is directed towards a specific reactor assembly, e.g. 1201a, only during a photochemical deprotection reaction step. Then, the controlled light pattern 1204b is directed towards other reactor assembly, while reactor assembly 1201a goes through the rest of synthesis steps, such as flushing, coupling, capping, etc.

The present invention is not limited to stepping mechanisms as discussed above. One example is a step-and-repeat exposure scheme, routinely used in photolithography of semiconductors. General descriptions of step-and-repeat photolithography are generally available. Thompson et al., In: Introduction to Microlithography, American Chemical—Society, Washington-D.C. (1994) Briefly, a large substrate, mounted on an x-y translation stage, contains multiple reaction-well arrays. At each step, a projected controlled light pattern covers one or several arrays. Then, the substrate is moved to the next position and another projected controlled light pattern is performed. The process is repeated until all reaction-well arrays are exposed.

The present invention is not limited to the use of electronic optical modulators as the means of generating photolithography patterns. Conventional photomasks, which are made of glass plates coated with patterned chromium or any other appropriate films, may be used as well. For example, the transmissive LCD display device 841 shown in FIG. 8C may be replaced with a conventional photomask. The use of conventional photomasks is preferred for the production of a large number of the same products. A conventional photomask may contain a large number of array patterns so that a large number of molecular arrays can be synthesized in parallel. However, for small batch production of various different array products the use of electronic optical modulators are preferred due to their flexibility.

Reactor Assembly Configuration

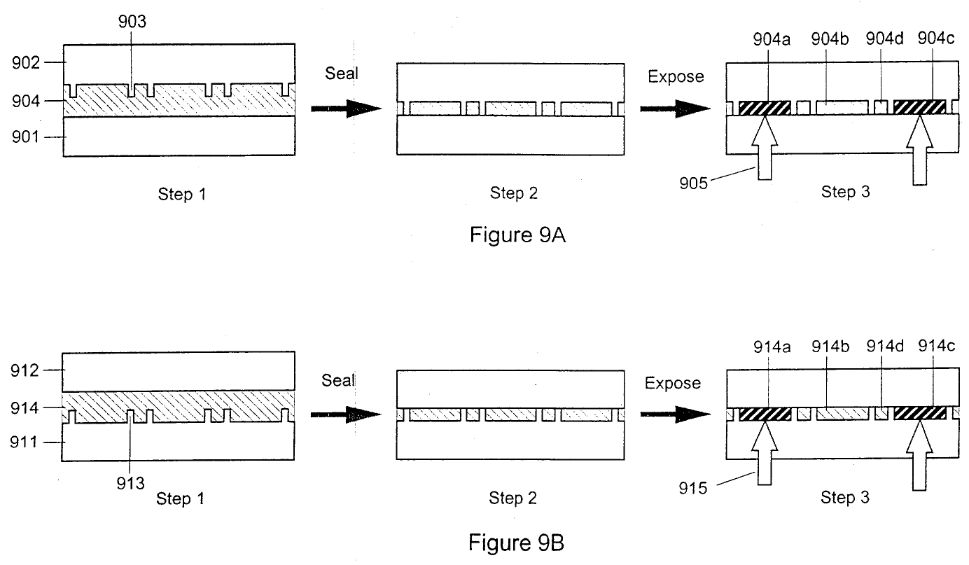

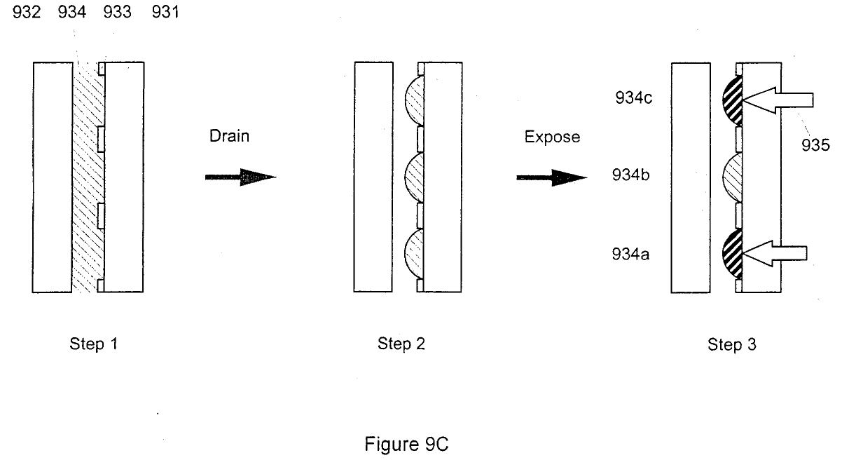

As described in earlier sections, one embodiment of the present invention contemplates photogenerated reagents (PGRs) in solution. When PGRs are used to produce a plurality of different sequences on the same solid substrate appropriate measures should be taken to spatially isolate the different sequences. FIGS. 9A through 9C schematically illustrate three preferred embodiments of isolation mechanisms contemplated by the present invention. In one embodiment, shown in FIG. 9A, a transparent solid substrate 901 and a cap 902 form a reactor assembly, which is filled with a solution containing one or more PGRPs. Within said reactor assembly are reaction-wells 904 bounded by embossed barriers 903 on a cap 902. Cap 902 is preferably made of a plastic or an elastomer material inert to all chemicals involved in the reaction. Before a photolytic reaction takes place, cap 902 is pushed against the solid substrate 901 forming contacts between the barriers 903 thus sealing and isolating said reaction-wells. A controlled light pattern is then projected into a plurality of isolated reaction-wells 904a and 904c, as shown in Step 3 of FIG. 9A. Photolytic and other PGR mediated reactions take place in the illuminated isolated reaction-wells 904a and 904c while there is no PGR mediated reactions occur in the non-illuminated reaction-well 904b. In a preferred embodiment, said isolated reaction-wells prevent diffusion of reagents between individual isolated reaction-wells. In addition, the space between adjacent isolated reaction-wells 904b and 904c provides a buffer zone 904d to further prevent any inter-mixing between isolated reaction-wells.

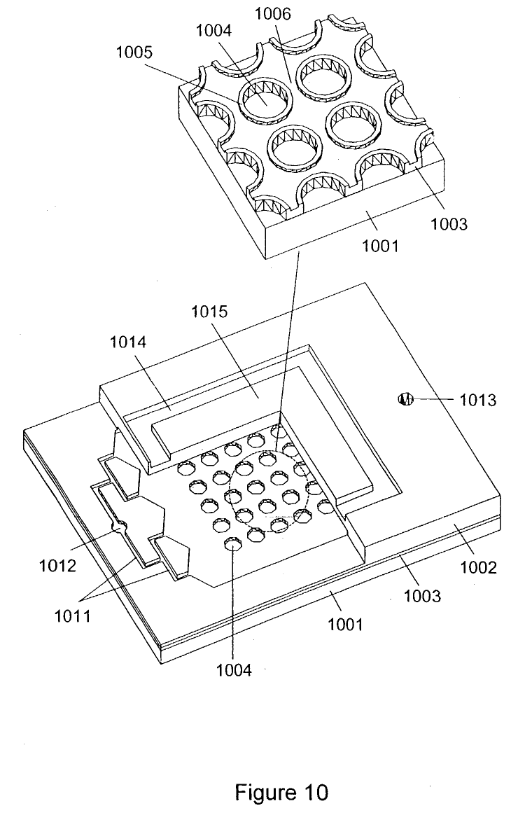

Other embodiments of the present invention contemplate that buffer zone 904d, shown in FIG. 9A, provides space for additional mechanisms of preventing cross-contamination among individual isolated reaction-wells. For example, FIG. 10 illustrates detailed structure of exemplary isolated reaction-wells of the current invention in a three-dimensional perspective view. The figure shows that all buffer zones (see 1006 in FIG. 10) are interconnected. This interconnected structure permits one to flush the buffer zone with appropriate solutions while all isolated reaction-wells are sealed. In one preferred embodiment, buffer zone 904d is flushed with a solution after the completion of the photolytic and PGR mediated reactions and before the lifting of the cap 90. Preferably, said solution quenches the PGR mediated chemical reactions or neutralizes excess PGRs remaining outside said illuminated isolated reaction-wells 904a and 904c. This neutralization prevents any spillover of excess PGRs surrounding the illuminated isolated reaction-wells 904a and 904c from causing undesirable chemical reactions in other areas (i.e., nonilluminated isolated reaction wells) after the cap 902 is lifted. For example, to neutralize an acid PGR, a weak basic solution, such as pyridine in CH₂Cl₂, may be applied. Alternatively, for quenching nucleotide coupling reactions, acetonitrile or other suitable solvents may be used.

FIG. 9B illustrates another embodiment of an isolation mechanism contemplated by the present invention. In this embodiment, isolated reaction-wells 913, are constructed on a transparent solid substrate 911 while cap 912 has a flat inner surface. Solid substrate 911 is preferably made of glass. Cap 912 is preferably made of a plastic or an elastomer material inert to all chemicals involved in the reactions. The sealing mechanism and the preferred operation mode are similar to those described earlier for the embodiment shown in FIG. 9A.

FIG. 9C illustrates a third embodiment of an isolation mechanism contemplated by of the present invention. In this embodiment, a pattern of non-wetting film 933 is coated on the surface of a transparent solid substrate 931. During an operation, a reactor assembly comprising said transparent solid substrate 931 is first filled with a solution 934. Then solution 934 is drained from the reactor assembly resulting in the formation of isolated droplets on solid substrate 931 because the solution 934 fails to adhere to said non-wetting film 934. A controlled light pattern 935 is projected onto transparent solid surface 931 thus illuminating droplets 934a and 934c to initiate photolytic and other PGR mediated reactions. This embodiment is particularly advantageous by eliminating the need for a sealing mechanism and is suitable for large-scale microarray or biochip production using large substrates. The use of non-wetting films to confine fluid is well-known in the art and has been described by Thomas M. Brennan in U.S. Pat. No. 5,474,596 for the synthesis of DNA oligomers using an inkjet-printing method.

In another embodiment contemplated by the present invention isolated reaction wells are integrated into a reactor cartridge assembly (see FIG. 10). The design shown in the figure utilizes, but is not limited to, the isolation mechanism shown in FIG. 9B. Other isolation mechanisms, such as the ones shown in FIGS. 9A and 9C, can be easily implemented into similar cartridge forms. As shown in FIG. 10, each cartridge contains a transparent solid substrate 1001, which can be made of glass or polymer materials of suitable chemical and optical properties. Above the solid substrate 1001 is a barrier layer 1003 containing a plurality of isolated reaction-wells 1004. In principle, isolated reaction-wells can be of any reasonable shapes and sizes, however, circular and square wells are preferred. In a preferred embodiment, said isolated reaction-wells are of circular shape ranging between 10-1,000 μm in diameter and 5-100 μm in depth. For example, in one specific embodiment, circular reaction-wells are 140 μm in diameter, 20 μm in depth, and are arranged orthogonally with equal center-to-center distances of 200 μm resulting in 2,500 reaction-wells per square centimeter. In each reaction-well, a sequence of approximately 6.4 fmol may be synthesized, assuming the average distance between immobilized adjacent sequences is 20 Angstroms. A sufficient volume for performing the synthesis reactions within said reaction-wells is about 300 pico-liter.

The barrier layer 1003 is made of opaque materials, such as metals or blackened polymers and prevents light-leakage between isolated reaction-wells. Reaction-well cap 1002 has three functions: reactor assembly enclosure, reagent connection and distribution, and reaction-well isolation. Cap 1002 is preferably made of a polymer material that is flexible and resistant to chemicals and solvents involved in any desired synthesis processes. The polymer material may be selected from a group comprising, but not limited to, polyethylene, polypropylene, polyethylene-polypropylene copolymer, fluorinated polymers and various other suitable ones. The reagent inlet 1012 and outlet 1013 are placed at two opposite ends of the reactor assembly. Branching channels 1011 are made to distribute reagents evenly across the reactor assembly. The center region of the cap is a pad 1015 that can be pushed down to tightly seal the isolated reaction-wells 1004. Immediately above the reactor assembly there is a mechanical actuator (not shown in FIG. 10 but shown in FIGS. 8A through 8C as 811, 831, and 851), which can be, for example, driven either solenoidally or pneumatically. The mechanical actuator can either push the pad of the reactor assembly top to seal all reaction-wells or retract to open all the reaction-wells. This operation is consistent with the exemplary sealing mechanism shown in FIGS. 9A and 9B. In one embodiment, said reaction-wells contain extruded rims 1005 to facilitate sealing (see FIG. 10, inset). While not shown in FIG. 10, the reactor assembly contains alignment marks, which permit alignment with an optical lithography system.

The reactor cartridge assembly shown in FIG. 10 is most suitable for use in an ordinary chemical and biochemical laboratory environment. The enclosed construction of said reactor cartridge assembly prevents chemical and particulate contamination from the environment. In order to achieve the best and consistent results, the cartridges are preferably manufactured in a controlled environment to ensure the chemical integrity inside the cartridge. The reactor cartridge assemblies are then filled with an inert gas, such as Argon, and sealed by plugging the inlets and outlets. Then the reactor cartridge assemblies can be stored and/or shipped to user laboratories.

Reaction-Well Fabrication

The various reaction-wells contemplated by the present invention (FIGS. 9A through 9C and 10) can be fabricated using various well-known microfabrication processes, such as photolithography, thin film deposition, electroplating, and molding (M. Madou, Fundamentals of Microfabrication, CRC Press, New York, (1997)). These techniques have been widely used for making various types of microfluidic devices, electromechanical devices, chemical sensors, and optical microdevices. For example, the reaction-well shown in FIG. 10 can be fabricated by using electroplating of suitable metal films on a glass solid substrate. The reaction-wells contemplated for glass substrates may also be made using chemical etching processes, which have been widely used to make various microfluidic devices (Peter C. Simpson et al., Proc. Natl. Acad. Sci., 95: 2256-2261 (1998)).

Reaction-well cap 1002, shown in FIG. 10, can be fabricated using a precision molding process. Such a process is widely available in plastic fabrication industry. The polymer material used is preferably in black color to minimize light reflection and scattering during light exposure. Welding and adhesive bounding methods can be used to assemble the plastic reaction-well cap 1002 and a solid substrate 1001 into an integrated reactor cartridge assembly.

Making non-wetting film patterns on glass and other solid substrates is a well-known art in many fields. Srinivasan et al., Proc. IEEE Solid-State Sensors and Actuators, pp. 1399-1402 (1991) The film is usually formed by a monolayer of self-assembled molecules (SAM) or a thin polymer film of low surface energy material such as Teflon. The most frequently used SAMs on glass solid substrates include various hydrocarbon alkylsilanes and fluoroalkylsilanes, such as octadecyltrichlorosiliane and 1H, 1H, 2H, 2H-perfluorodecyltrichlorosiliane. The patterning process involves the use of photoresists and photolithography (see Example VII) Thin polymer films, such as Teflon, can be printed onto glass and plastic surfaces by using a screen printing process. The screen printing process is a well-know art in printing industry and in electronic industry. General procedures of screen printing for microfabrication applications are described by M. Madou in Fundamentals of Microfabrication, CRC Press, New York, (1997). In addition, hydrophobic printed slides are commercially available from vendors, such as Erie Scientific Company, Portsmouth, N.H. USA. When non-wetting film patterned substrates are used, the reactor assembly configuration can be simplified because the reaction-well sealing mechanisms shown in FIGS. 8A through 8C and FIG. 10 are no longer needed.

Control of the Apparatus

As illustrated in FIG. 8A, the synthesis apparatus of the present invention is controlled by a computer 814, which coordinates the actions of the DMD 801, the seal actuator 811 of the reactor assembly 810, and the reagent manifold 812. In case of synthesizing oligonucleotides, during most of synthesis steps, the synthesis apparatus operates as a conventional synthesizer and the computer 814 controls reagent manifold 812 to deliver various reagents to the reactor assembly 810. At a step involving the projection of a controlled light pattern, the reagent manifold 812 delivers an acid PGRP into the reactor assembly 810. The computer 814 activates the seal actuator 811 to isolate reaction-wells 810a and, then, sends data to DMD 801 to project a controlled light pattern 807c onto the reactor assembly 810. At the completion of the photoreactions, the controlled light pattern 807c is switched off, a quenching solution is delivered into the reactor assembly 810, the seal actuator 811 is lifted, and the synthesis control system resumes the steps of conventional synthesis.

Variations and Modifications

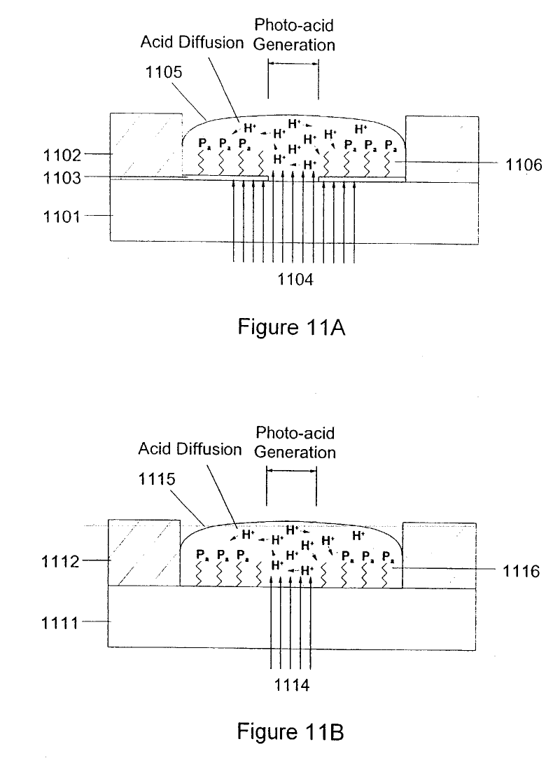

The present invention contemplates many possible variations and applications. For example, FIG. 11A illustrates a reaction-well variation wherein a mask layer 1103 is added to the bottom of the reaction-well. One or more openings, which occupy a total one tenth to one half of the reaction-well surface area, are made on the masks for a controlled light pattern 1104 to pass through. The mask layer 1103 is preferably made of a thin and chemical resistant metal film, such as chromium. On top of the metal film, an SiO₂ film (not shown) is deposited to facilitate immobilization of linker molecules. This isolated reaction-well design permits an exemplary spatial separation of a photochemical reaction and PGR mediated chemical reactions. FIG. 11A illustrates an acid PGR mediated chemical reaction. Upon projection of a controlled light pattern, protons (H⁺) are produced from an acid PGRP in the illuminated areas. These protons, then, diffuse throughout the isolated reaction-well to cleave acid-labile protecting groups Pₐ on immobilized oligomer molecules 1106. This variation minimizes the contact between photo-generated radical intermediates and the oligomers and thus, suppresses undesirable side-reactions.

FIG. 11B illustrates another variation of isolated reaction-wells and projection of controlled light patterns. This one embodiment decreases the possibility of undesirable side reactions due radical intermediates. Since only a fraction of the isolated reaction-well is exposed to a controlled light pattern 1114, the chance for undesirable side-reactions in other areas is decreased.

Potential embodiments of the chemical processes and the apparatus (FIGS. 8A through 8C) contemplated by the present invention extend beyond the fabrication of biopolymer sequences. For example, the apparatus using DMD 801 shown in FIG. 8A may be used as a general-purpose assay apparatus for studying chemical and biochemical reactions. The Digital Micromirror Device 801 controls precisely and simultaneously light patterns in all isolated reaction-wells of a reactor assembly 810. The DMD thereby allows precise control of the production PGRs in all reaction-wells and, therefore, facilitates the performance of large-scale, parallel assay.

Obviously many modifications and variations of this invention are possible in the light of the above teachings. It is therefore to be understood that within the scope of the appended claims the invention may be practiced otherwise than as specifically described.

III. Microarray or Biochip Platforms

Miniaturization of conventional experiments to a microarray or biochip platform reduces reagent consumption, minimizes reaction volumes, increases the sample concentration, and accelerates the reaction kinetics. Schena, M., R., Heller, A., Theriault, T. P., Konrad, K., Lachenmeier, E., Davis, R. W., Microarrays: Biotechnology's Discovery Platform For Functional Genomics. Trends Biotechnol. 16, 301-306 (1998). The microarray platform allows parallel synthesis of molecular libraries, massively parallel assaying, and data acquisition. Of the various libraries of bioorganic compounds, those comprising peptides have increasingly been used as useful tools in all areas of biomedical research. They have been successfully employed to study antibody-antigen interactions, to develop enzyme inhibitors and antimicrobials, and to engineer novel properties in antibodies. Houghten, R. A., Soluble Chemical Combinatorial Libraries: Current Capabilities And Future Possibilities, In Peptides: Synthesis, Structure And Application; Gutte, B., Ed.; Academic Press:CA, pp 395-417 (1995).; Rodda et al., Epitope Mapping, In: Combinatorial Peptide And Non-peptide Libraries, Jung, G., Ed.; VCH:New York, pp 303-326 (1996).

The present invention contemplates two main methods of making peptide microarrays or biochips.

Method 1: Oligopeptide Presynthesis: In one embodiment presynthesized oligopeptides are transferred to a solid substrate. Preferably, this transfer is selected from the group consisting of mechanical microspotting and piezoelectric delivery. Specific advantages of this particular embodiment involves rapid implementation with high versatility and low cost. In another embodiment, very large numbers of oligopeptides are synthesized, purified, and stored prior to microarray or biochip fabrication.

Method 2: Light-Labile Protected Amino Acid Photolithography: In one embodiment the present invention contemplates the simultaneous synthesis of different peptide oligomers directly on a solid substrate. The first generation of the peptide microarray of this type was developed by the Affymetrix group. Fodor et al., Light-Directed, Spatially Addressable Parallel Chemical Synthesis, Science, 251:767-773 (1991). In this previous method the locations of the synthesis for different peptides are controlled by a series of photolithographic masks, which are expensive, time-consuming to build and design, and are severely limited in their application.

Various embodiments of the present invention contemplate alternative, high yield and low cost methods, to fabricate peptide microarrays or biochips. These embodiments are an improvement on previous synthesis methods of oligopeptides using BOC chemistry and a micromirror protection system. Pellois et al., Peptide Synthesis Based On t-Boc Chemistry And Solution Photogenerated Acids, J. Comb. Chem. 2:349 (2000).

One aspect of the present invention contemplates conventional chemistry liquid-phase synthesis using conventional acid deprotection of amino acids. Another aspect of the present invention contemplates using spatially-directed generation of acids with a laser scanner for parallel synthesis of peptides.

In one embodiment conventional acid deprotection results in a high efficiency of the peptide synthesis. Preferably, acid PGR is obtained by exposing an acid PGRP to light at its absorption wavelength. In another embodiment, deprotection of compounds comprising acidlabile protecting groups occurred only in illuminated isolated reaction-wells following the conversion of acid PGRP into acid PGR. Leproust et al., Digital Light-Directed Synthesis. A Microarray Platform That Permits Rapid Reaction Optimization On A Combinatorial Basis, J. Comb. Chem. 2:349-354 (2000). In another embodiment, the present invention contemplates the synthesis of a plurality of different peptides simultaneously synthesized in a plurality of different isolated reaction wells on the solid substrate (i.e., a microarray or biochip). To test the concept, a comparative synthesis of pentapeptide models was performed on glass microscope slides using conventional acid, trifluoroacetic acid, and acid PGR to remove the protection group from the peptide. The stepwise synthesis efficiency of the two chemical methods was quantified and was found to be identical within measurement accuracy. Then the light-directed parallel synthesis of oligopeptide models was performed on microarray substrates. The accuracy of the amino acid sequence in the oligopeptide models was tested by specific recognition binding.

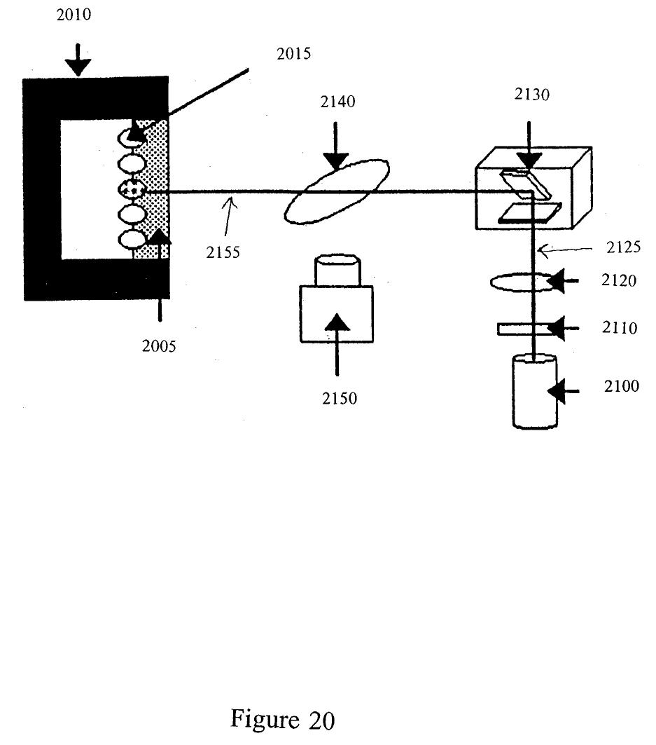

An exemplary UV illumination system is shown in FIG. 20. Preferably, the system comprises a light source 2100 (i.e., for example a laser diode (405 nm, 25 mW)), a pinhole 2110 and focusing lens 2120 to confine the size of the laser beam 2125, a laser scanner 2130 to project the laser beam to different locations, a beam splitter 2140, and a video camera 2150 to assist in the projection of a controlled light pattern 2155 onto isolated reaction-wells 2015 residing inside the reactor cartridge assembly 2010. The laser beam 2125 characteristics and projection of said controlled light pattern 2155 is synchronized and controlled by LASER DESIGNER AND SHOWTIME software.

One aspect of the present invention contemplates producing microarrays and biochips comprising glass. Preferably, protected amino acid monomers (i.e., for example, monomers comprising tert-butoxycarbonyl; BOC) having differing sequences are attached (i.e., spotted) onto separate areas of derivatized microarrays and biochips. Optionally, verification of proper linker attachment may be performed by labeling with 5,6-carboxyfluorescin (FAM). The resulting FAM fluorescence intensity is useful both as a positive control for the coupling reaction of protected amino acid monomers and as a negative control for the deprotection reaction. Specifically, if there are no free amino reactive terminal groups on a microarray or biochip, FAM will not bind. Any fluorescence intensity detected on an amino-free microarray or biochip is due to nonspecific binding (i.e., a background signal). One embodiment of the present invention contemplates using a derivatized microarray or biochip as a negative control for coupling reactions. Another embodiment of the present invention contemplates using a derivatized microarray or biochip as a positive control for deprotection reactions.

Another aspect of the present invention contemplates derivatizing microarrays and biochips. In one embodiment, derivatization comprises a linker attached to a microarray or biochip via silanol groups, preferably exposing free amino reactive terminal groups to the interior of the isolated reaction-well. In another embodiment, said exposed free amino reactive terminal groups represent a maximum number of attachment sites for amino acid monomers or FAM. In one embodiment, contacting FAM with a microarray or biochip comprising attached linkers results in an equivalent fluorescence intensity when compared to clean glass. In another embodiment, said equivalent fluoresence intensity of attached protected linkers to clean glass indicates a greater than 98% linker coupling efficiency (i.e., FAM will bind to unattached silanol groups). In one embodiment, deprotection by 50% TFA or 10% SSb with illumination results in equivalent fluorescence intensities. Although it is not required to understand the mechanism of the invention, it is believed that SSb with illumination produced acid PGRs that deprotected linkers as efficiently as TFA.

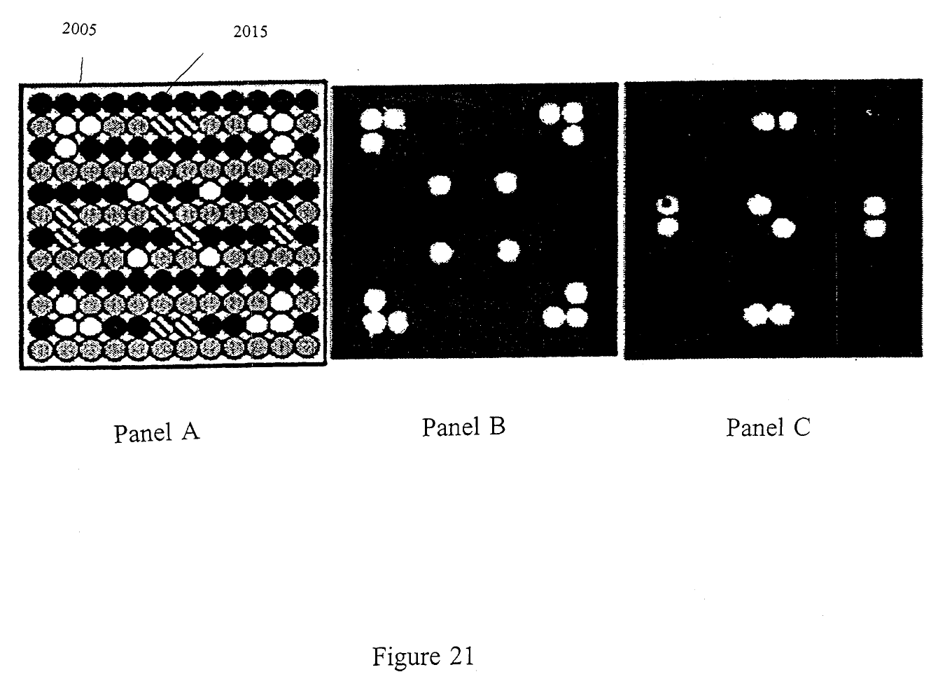

In another aspect of the present invention, variations in fluorescence intensity may be avoided due to the use of different types of amino acids by using a glycine pentapeptide. Preferably, a glycine pentapeptide is used to obtain and compare stepwise efficiency. In one embodiment, a glycine pentapeptide is synthesized by either SSb or TFA deprotection wherein following each glycine monomer attachment (i.e., forming a multimer) said multimer is contacted with FAM. Preferably, fluorescence intensity of most recently coupled glycine is compared with fluorescence intensity of the previous cycle wherein a stepwise efficiency ratio may be calculated. In one embodiment, said stepwise efficiency ratio includes both coupling efficiency and deprotection efficiency. In another embodiment, said stepwise efficiency ratio reflects deprotection when both coupling steps are performed using the same acid deprotection method. In another embodiment, stepwise efficiency ratios are equivalent when using either SSb or TFA. (see FIG. 21) Preferably, stepwise efficiency ratios range between 80% and 100%. In another embodiment, chain length of a peptide does not effect the value of stepwise efficiency ratios. Sarin et al., A General Approach To The Quantitation Of Synthetic Efficiency In Solid-Phase Peptide Synthesis As A Function Of Chain Length, J. Am. Chem. Soc. 106:7845-7850 (1984).

In another aspect of the present invention it is contemplated that acid PGR chemistry is combined with a controllable light illumination system (i.e., for example, a system that projects a controlled light pattern). In one embodiment the present invention contemplates the the parallel synthesis of different oligopeptides on a microarray or biochip platform. In another embodiment, the present invention contemplates the simultaneous synthesis on a microarray or biochip of at least four oligopeptides selected from the group consisting of dns-Glu-Cys-Glu-Glu, H₂N-Glu-Glu-Glu-Glu, dns-Cys-Cys-Cys-Cys, and dns-Gly-Gly-Gly-Gly. Preferably, the first peptide analogue has specific Pb²⁺ binding capability wherein said binding is detected by an increase in fluorescence intensity. Deo et al., A Selective, Ratiometric Fluorescent Sensor For Pb²⁺, J. Am. Chem. Soc., 122:174-175 (2000). Alternatively, peptide analogues are simultaneously synthesized that do not bind with Pb²⁺.

Another aspect of the present invention contemplates the detection of a specific oligonucleotide sequence following simultaneous synthesis of a plurality of different sequences. In one embodiment, a microarray or biochip is produced by the simultaneous synthesis of a plurality of oligopeptides as exemplified in FIG. 21. Possible locations for isolated reaction-wells to synthesize each oligopeptide are shown in FIG. 21 Panel A. In one embodiment, a microarray or biochip comprising a plurality of simultaneously synthesized oligopeptides are contacted with 1 mM PbCl₂ in buffer solution. In another embodiment, fluorescence intensity is detected from oligonucleotides binding Pb²⁺. In a preferred embodiment, fluorescence intensity is greatest at isolated reaction-wells comprising Pb²⁺ binding oligopeptides (i.e., for example, see FIG. 21 Panel B). In another embodiment, a significant increase in fluorescence intensity is observed at isolated reaction-wells comprising an oligopeptide containing the sequence dns-Glu-Cys-Glu-Glu. In an alternative embodiment, an increase is fluorescence is not observed at isolated reaction-wells comprising an oligonucleotide containing a sequence selected from the group consisting of H₂N-Glu-Glu-Glu-Glu, dns-Cys-Cys-Cys-Cys and das-Gly-Gly-Gly-Gly. Although it is not necessary to understand the mechanism of the present invention, it is believed that the variations in detected fluoresence intensity indicate the isolated reaction-wells that correspond to the correct locations of each simultaneously synthesized oligopeptide.

Another aspect of the present invention contemplates a method of screening for a new target peptide capable of binding As³⁺. In one embodiment, a microarray or biochip comprising a plurality of oligopeptide analogues selected from the group consisting of dns-Glu-Cys-Glu-Glu, H₂N-Glu-Glu-Glu-Glu, dns-Cys-Cys-Cys-Cys, and das-Gly-Gly-Gly-Gly, wherein at least one of said oligopeptide analogues is capable of binding As³⁺ (i.e., for example by contacting said oligopeptide analogues with NaAsO₂⁻). In a preferred embodiment, fluoresence intensity is greatest at isolated reaction-wells comprising a sequence capable of binding As³⁺. In another embodiment, fluoresence intensity is greatest when As³⁺ is bound to a sequence comprising dns-Glu-Glu-Glu-Glu (see FIG. 21 Panel C). This particular embodiment of this invention contemplates novel As³⁺-binding oligopeptides that are useful as biosensors.

Binding for both metal ions is also observed following a separate synthesis of peptide analogues on different glass microscope slides using conventional trifluoroacetic acid procedures (data not shown). Increases in fluorescence intensity with respect to each peptide analogue is in agreement with various embodiments of the present invention utilizing peptide microarrays and biochips. Consequently, the present invention contemplates a device and method for parallel peptide synthesis using acid PGR chemistry and a laser diode/scanner system based on microarrays and biochips having high accuracy in regards to both oligopeptide sequence and synthesis location.

Previous methods have produced immunoassay peptide librarys based on conventional photolithographic masking techniques. Fodor et al., Method For Screening Receptor-Ligand Binding Using Probe Arrays, U.S. Pat. No. 6,124,102 (2000). In this study, the specific binding of hexapeptide Ser-Asp-Leu-His-Lys-Leu (SDLHKL), which is the amino acid residue 20-25 of human p53 protein, and MAb DO-1 were selected as the model for application of immunoassaying. Stephen et al., Characterisation Of Epitopes On Human p53 Using Phage-Displayed Peptide Libraries: Insights Into Antibody-Peptide Interactions, J. Mol. Biol., 248:58-78 (1995). This method requires the cumbersome and expensive techniques refers to above that limits the production capability of standard photolithography.

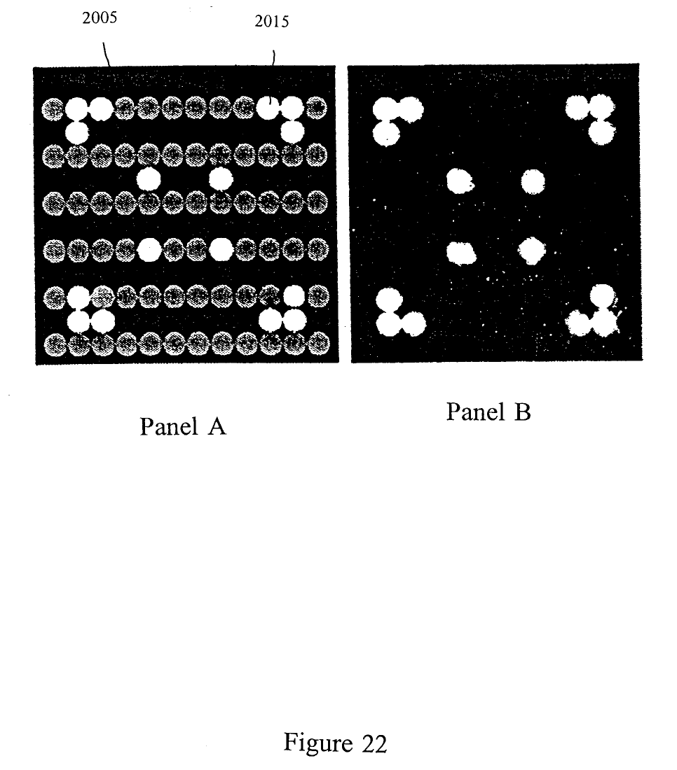

Another aspect of the present invention contemplates the creation of an immunoassay peptide library by the simultaneous synthesis of a plurality of oligopeptides on a microarray or biochip using acid PGR chemistry and a laser diode/scanning system (see FIG. 22 Panel A). In one embodiment, the present invention contemplates the simultaneous synthesis of oligopeptides comprising SDLHKL, DSLGKL and SGLHKL attached to said microarray or biochip within different isolated reaction-wells. In another embodiment, a plurality of simultaneously synthesized oligopeptides are probed with mouse monoclonalantibody DO-1 directed against SDLHKL (i.e., for example, amino acids 20-25 of human peptide p53) and contacted with a fluoresceinated rabbit anti-mouse antibody. In a preferred embodiment, fluorescence intensity is detected at isolated reaction-wells comprising an oligopeptide comprising an amino acid sequence SDLHKL (see FIG. 22 Panel B). In another embodiment, fluorescence intensity is not detected at isolated reaction-wells comprising an oligopeptide, wherein said oligopeptide comprises an amino acid sequence differing from SDLHKL by only a single amino acid. In a preferred embodiment, fluorescence intensity is not detected from an oligopeptide comprising an amino acid sequence differing from SDLHKL by a single amino acid selected from the group consisting of DSLGKL and SGLHKL.

Although it is not necessary to understand the mechanism of the invention, it is believed that oligopeptides synthesized by acid PGRs and laser diode/scanner system on microarrays and biochips are biologically recognized. Additionally, fluoresence probe analysis following the synthesis of hexapeptides on separate glass slides using conventional acid, 50% trifluoroacetic acid show similar binding as oligopeptides simultaneously synthesized on microarrays and biochips. Consequently, the present invention contemplates an accurate and reliable synthesis of oligopeptides that differ in amino acid sequence by synthesis in isolated reaction-wells.

Another aspect of the present invention contemplates an extremely high deprotection efficiency. In one embodiment, simultaneously synthesized polypeptides of glycine attached to microarrays or biochips at different isolated reaction-wells are identified by fluoresence intensity (see FIG. 23). Specifically, FIG. 23 illustrates the expected fluoresence intensity after the deprotection step of each synthesis cycle. In one embodiment, the fluoresence intensity remains approximately the same after each glycine deprotection during the synthesis of a glycine pentapeptide. The stepwise synthesis efficiency (supra) was calculated from the ratio of fluorescence intensity of a certain step to that of its previous step. In a preferred embodiment the present invention contemplates stepwise synthesis efficiencies consistently ranging from approximately 96-100%.

The above description demonstrates one alternative technique for the fabrication biopolymer microarrays or biochips. Specific advantages of this system rely on the chemistry of acid PGRs and maskless light illumination of isolated reaction-wells. Using this combination, the simultaneous synthesis of different oligopeptides at different isolated reaction-wells is accurate and reliable as well as simple and convienent.

Experimental

The invention is further described by the following Examples, which are provided for illustrative purposes only and are not intended nor should they be construed as limiting the invention in any manner. Those skilled in the art will appreciate that variations on the following Examples can be made without deviating from the spirit or scope of the invention.

### Example I

### Production of Acid Photogenerated Reagent

This experiment demonstrates efficient generation of H⁺ upon illumination of an acid PGR as monitored by an increased value in the chemical shift of the H₂O signal as a function of illumination time.

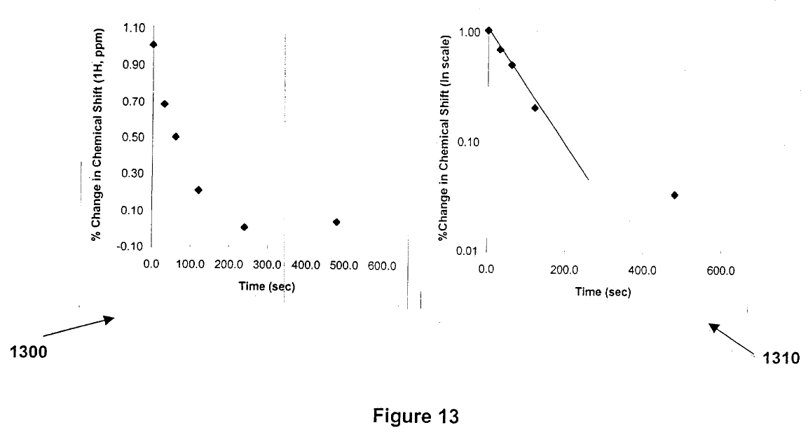

Six samples containing a sulfonium salt (0.4% of 50% triaryl sulfonium hexaflurophosphate in propylene carbonate, Secant Chemicals, Boston, Mass.) in. 0.5 mL CD₂Cl₂ were placed in nuclear magnetic resonance (NMR) tubes. A reference one-dimensional (ID) spectrum of these samples was recorded (600 MHz NMR spectrometer, Bruker, Karlsruhe, Germany) using a method well known to those skilled in the art. One of the samples was then illuminated using a collimated light source (22 mW, Oriel, Stanford, Calif.) at 365 nnm for a defined length of time (FIG. 13) and a 1D NMR spectrum was recorded immediately. A second sample was then illuminated at 365 nm for a second defined length of time (FIG. 13) and a 1D NMR spectrum was recorded immediately. These experiments were repeated for each of the samples prepared. For each NMR spectrum, the chemical shift of the H₂O was measured. In the absence of illumination, the H₂O signal appeared at 1.53 ppm. In the presence of illumination, this signal moved to a higher ppm value (down field shifted) due to the generation of H⁺.

In FIG. 13, the correlation of the changes in chemical shift of the H₂O signal with illumination time is plotted. The formation of H⁺ under the conditions used follows a first order kinetics relationship and the apparent rate constant for formation of H⁺ derived is 1.3×10⁻²±0.06 s⁻¹.

### Example II

### Deprotection of Nucleoside Monomers using Acid PGR

These experiments demonstrate efficient deprotection of the DMT group on 5′-OH of nucleosides using an acid PGR.

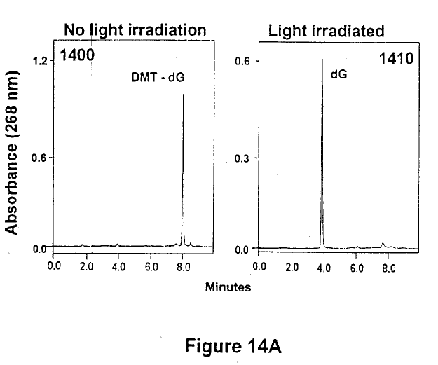

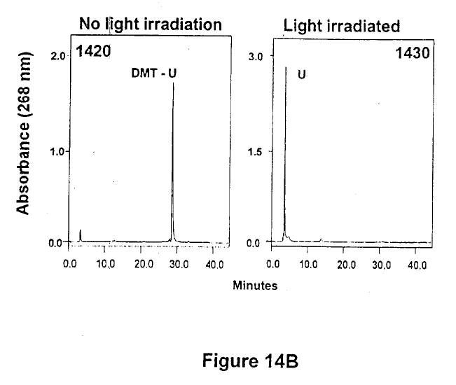

Two samples were prepared in which DMT-G attached to (controlled porous glass, 0.2 pmol (CPG) added to sulfonium salt (0.4% of 50% triaryl sulfonium hexaflurophosphate in propylene carbonate, Secant Chemicals, Boston, Mass.) in 0.5 mL CH₂Cl₂. One sample was illuminated using a UV lamp (UVGL-25, 0.72 mW) at 365 nm for 2 min, while the other sample, a control, was not illuminated. Upon completion of the illumination, CPG was washed with CH₂Cl₂ and CH₃CN, followed by treatment with concentrated aqueous NH₄OH (1 mL) for 2 h at 55° C. The solution was briefly evaporated under vaccuum. A buffer solution (0.1 M triethylammonium acetate (TEAA), 15% in CH₃CN) was added to the CPG sample and the resultant solution was injected into a C18 reverse phase (10 pm, μ-bondapak, Waters) HPLC column. A gradient of 0.1 M TEAA in CH₃CN was used to elute the sample. Authentic samples of DMT-dG and dG were used as reference and co-injection of acid PGR deprotected dG and authenic dG confirms the result of the acid PGR reaction. 1400 and 1410 of FIG. 14A show HPLC profiles of DMT-dG and the acid PGR deprotected dG.

The same procedures were performed for DMT-dC, DMT-dG, DMT-dA, and DMT-rU. 1420 and 1430 of FIG. 14B show HPLC profiles of DMT-rU and the acid PGR deprotected rU.

Other acid PGRPs, such as 2,1,4-diazonaphthoquionesulfonate triester, triaryl sulfonium hexafluroantimonate and hexaflurophosphate (Secant Chemicals, Boston, Mass.), and perhalogenated triazine (Midori Kagaku), were also used for these deprotection reactions. Complete deprotection of the DMT group was achieved with these acid PGRPs.

### Example III

### Deprotection of Nucleoside Monomers using Pre-Activated Acid PGR

This experiment demonstrates that pre-activation of acid PGRP is an effective means of reducing side reactions in deprotection using acid PGRs. Depurination due to cleavage of glycosidic bonds in nucleotides under acidic conditions is a known problem. This problem is exacerbated in the use of acid PGRs for deprotection since at the initiation of reaction, the amount of H⁺ requires time to build up. The following experiment is to show that this problem can be alleviated using a pre-activated acid PGRs.

The samples and experimental conditions used in this experiment were as described in Example II, except that the acid PGR solution (0.4% of 50% triaryl sulfonium hexafluroantimonate in propylene carbonate) was first illuminated at 365 nm for 2 minutes before adding the CPG attached DMT-nucleoside.

Pre-illumination (UVGL-25, 0.72 mW) at 365 nm for 2 minutes was performed using an acid PGR solution (0.4% of 50% triaryl sulfonium hexafluroantimonate in propylene carbonate). The illuminated solution was then added to powder DMT-dA (approximately 1 pmol). The solution was incubated for an additional 2 minutes and a 1D NMR spectrum was recorded using methods well known to those skilled in the art. Another sample of DMT-dA (1 pmol) was mixed with an acid PGR solution (0.4% of 50% triaryl sulfonium hexafluroantimonate in propylene carbonate) and the mixture was illuminated (UVGL-25, 0.72 mW) at 365 nm for 2 minutes and a 1D NMR spectrum was recorded. Depurination causes gradual disappearance of the signals of dA. The comparison of the two NMR spectra recorded for these experiments indicates less side reactions using pre-activated acid PGR.

### Example IV

### Oligonucleotide Synthesis using Acid PGR

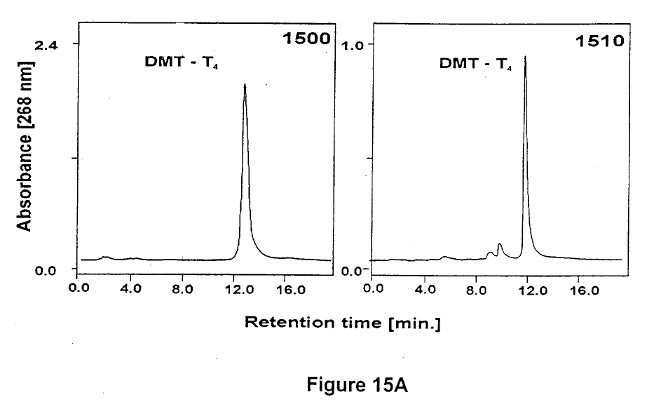

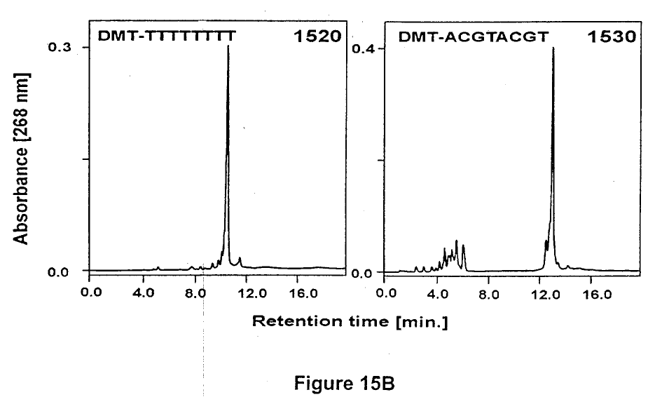

These experiments demonstrate efficient synthesis of oligonucleotides on CPG support using acid PGR. Oligonucleotides of various sequences (A, C, G, and T) and chain lengths (n=2-8) were synthesized using acid PGRPs of a Perspective Synthesizer (Perspective Biosystems, Framingham, Mass.). Synthesis of DMT-TTTT (1510 of FIG. 15), was carried out on a 0.2 μmol scale according to the protocol in Table 2. This is a direct adoption of the conventional phosphoramidite synthesis but with minor modifications at step 2. At this step, an acid PGR (0.4% of 50% triaryl sulfonium hexafluorophosphate in propylene carbonate) was added and the reaction column was illuminated with 365 nm light for 2 minutes. The column was extensively washed with solvents after the acid PGR mediated deprotection reaction. Upon completion of the synthesis, the sequence was cleaved from CPG and deprotected using concentrated NH₄OH. The sample was examined using C18 reverse phase HPLC using a TEAA in CH₃CN gradient. The HPLC profile of the crude product of DMT-TTTT synthesized using an acid PGR is shown (1510 of FIG. 15A). 1500 of FIG. 15 shows DMT-TTTT using conventional TCA deprotection chemistry. 1520 and 1530 of FIG. 15B show HPLC profiles of the crude octanucleotides which were synthesized using the acid PGR approach.

TABLE 2A
Protocol Of Automated Oligonucleotide Synthesis
Using An Acid PGR (0.2 μM)¹

| | | | | | | Amt. |
|---|---|---|---|---|---|---|
| | | | Vol. | Time | Conc. | Used |
| | | | (ml) | (sec) | (mM) | (μmol) |
| 1 | detritylation² | 1% UVL-6974/CH₂Cl₂ (v/v) | 1.20 | 180 | 100 | 114 |
| 2a | wash | CH₃CN | 2.40 | 200 | | |
| 2c | wash | CH₂Cl₂ | 2.00 | 50 | | |
| 3 | coupling | A. tetrazole/CH₃CN | 0.10 | 2 | 450 | 45 |
| 4 | coupling | B. tetrazole/CH₃CN | 0.10 | 2 | 450 | 45 |
| 5 | (simultaneous) | B. monomer/CH₃CN | 0.10 | 2 | 100 | 10 |
| 6 | coupling | B. tetrazole/CH₃CN | 0.10 | 63 | 450 | 45 |
| 7 | wash | CH₃CN | 0.04 | 31 | | |
| 8 | wash | CH₃CN | 0.66 | 17 | | |
| 9 | capping | A. acetic anhydride/ | 0.15 | 4 | 10% | 147 |
| | | utidine/THF | | | | |
| 10 | (simultaneous) | B. N-methylimidazole/THF | 0.15 | 4 | 10% | 183 |
| 11 | wash | CH₃CN | 0.10 | 15 | | |
| 12 | wash | CH₃CN | 0.27 | 7 | | |
| 13 | oxidation | I₃/THF/pyridine/H₂O | 0.29 | 7 | 0.02 | 6 |
| 14 | wash | CH₃CN | 0.29 | 7 | | |
| 15 | capping | A. acetic anhydride/ | 0.13 | 3 | 127 | |
| | | utidine/THF | | | | |
| 16 | (simultaneous) | B. N-methylimidazole/THF | 0.13 | 3 | | 158 |
| 17 | wash | CH₃CN | 0.57 | 15 | | |
| | total (sec) | | | 612 | | |
| | total (mm) | | | 10.2 | | |

¹Protocol is adopted from an Expedite 8909 synthesizer used for oligonucleotide synthesis using acid PGR deprotection.
²Highlighted steps for incorporation of the acid PGR reactions. Controlled light pattern illumination is applied at this step.

TABLE 2B
Protocol Of Automated Oligonucleotide Synthesis Using Conventional TCA

| | | | | | | Amt. |
|---|---|---|---|---|---|---|
| | | | | Time | Conc | Used |
| | | | Vol. (ml) | (sec) | (mM) | (μmol) |
| 1 | detritylation | 3% TCA | 1.20 | 59 | 100 | 114 |
| 2 | wash | CH₂Cl₂ | 1.00 | 20 | | |
| 3 | coupling | A. tetrazole/CH₃CN | 0.10 | 2 | 450 | 45 |
| 4 | coupling | B. tetrazole/CH₃CN | 0.10 | 2 | 450 | 45 |
| 5 | (simultaneous) | B. monomer/CH₃CN | 0.10 | 2 | 100 | 10 |
| 6 | coupling | B. tetrazole/CH₃CN | 0.10 | 63 | 450 | 45 |
| 7 | wash | CH₃CN | 0.04 | 31 | | |
| 8 | wash | CH₃CN | 0.66 | 17 | | |
| 9 | capping | A. acetic anhydride/ | 0.15 | 4 | 10% | 147 |
| | | utidine/THF | | | | |
| 10 | (simultaneous) | B. N-methylimidazole/THF | 0.15 | 4 | 10% | 183 |
| 11 | wash | CH₃CN | 0.10 | 15 | | |
| 12 | wash | CH₃CN | 0.27 | 7 | | |
| 13 | oxidation | I₃/THF/pyridine/H₂O | 0.29 | 7 | 0.02 | 6 |
| 14 | wash | CH₃CN | 0.29 | 7 | | |
| 15 | capping | A. acetic anhydride/ | 0.13 | 3 | | 127 |
| | | utidine/THF | | | | |
| 16 | (simultaneous) | B. N-methylimidazole/THF | 0.13 | 3 | | 158 |
| 17 | wash | CH₃CN | 0.57 | 15 | | |
| | total (sec) | | | 261 | | |
| | total (mm) | | | 4.35 | | |

### Example V

### Amino Acid Deprotection and Oligopeptide Synthesis using Acid PGRs

These experiments demonstrate efficient deprotection of the amino protection group using acid PGRs in oligopeptide synthesis.

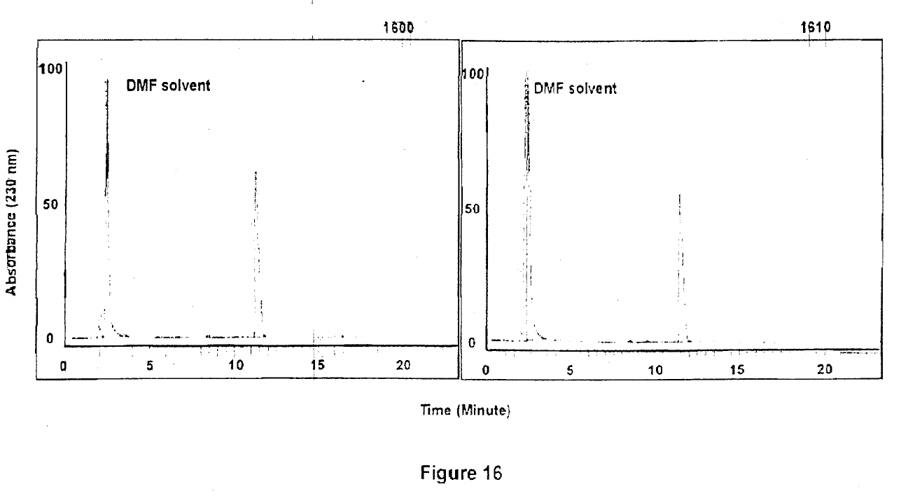

A sample of 10 mg (10 μmol) of HMBA resin (Nova Biochem, La Jolla, Calif.) containing t-Boc-Tyr was employed. Deprotection was performed in a CH,Cl, solution containing an acid PGR (10% of 50% triarylsulfonium hexafluroantimonate in propylene carbonate) by illuminating the same solution at 365 nm for 15 min. The reaction was incubated for an additional 15 min and the resin was washed with CH₂Cl₂. The possible presence of residual amino groups was detected using ninhydrin color tests and the result was negative. The resin was then washed and the amino acid cleaved from the resin using NaOH (0.1 M in CH₃OH). 1610 of FIG. 16 shows the HPLC profile of the acid PGR deprotected Tyr. 1600 of FIG. 16 shows the HPLC profile of Tyr obtained using conventional trifluoroacetic acid (TFA) deprotection.

Synthesis of a pentapeptide, Leu-Phe-Gly-Gly-Tyr, was accomplished using 100 mg of Merrifield resin. The acid PGR deprotection of the t-Boc group was performed and the resin was tested using ninhydrin until no color resulted. Coupling reactions were carried using conditions well known to those skilled in the art. The acid PGR deprotection and coupling steps were repeated until the pentamer synthesis was completed. The sequence was cleaved from the resin and its HPLC compared well to that of the same sequence synthesized conventional peptide chemistry.

### Example VI

### Fabrication of Microarrays and Biochips

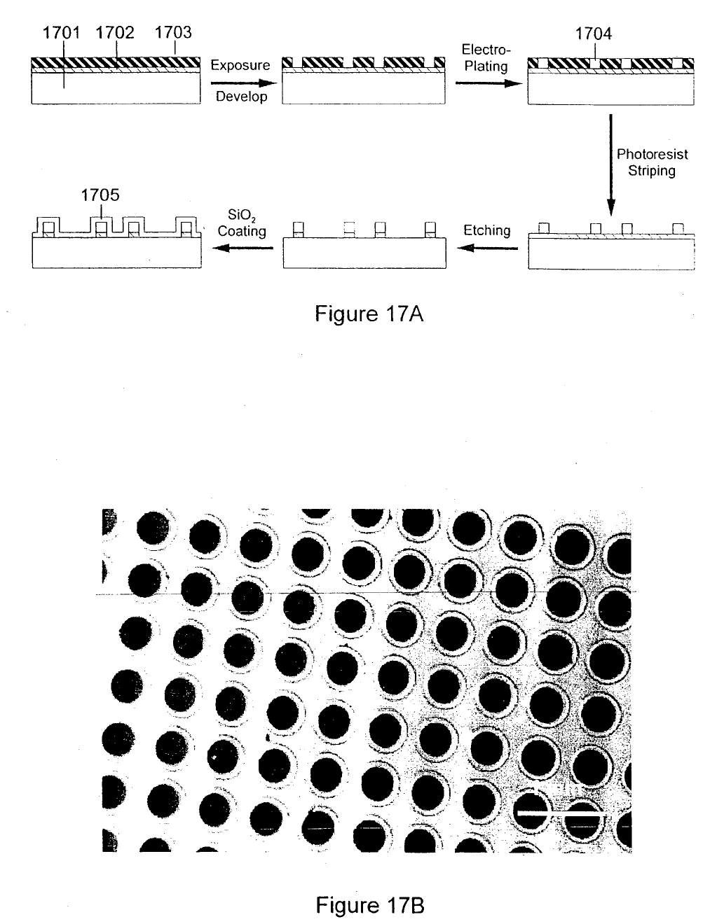

Formation of microarrays and biochips using fabrication methods contemplated by the present invention is demonstrated in this example. FIG. 17A schematically illustrates the fabrication procedure used. In the first fabrication step, a thin bimetal film 1702 of Cr/Cu 200/1000 Angstroms thick was evaporated on a glass substrate 1701 in a sputtering evaporator. The bimetal film 1702 Cr provides good adhesion to the glass surface and Cu provides a good base for subsequent electroplating. The surface was then spin-coated with a positive photoresist 1703 of 18 μm thick. The photoresist film 1703 was then patterned using photolithography (exposure to UV light using a photomask aligner and development). Electroplating using a plating solution for bright Ni was utilized to apply a plate a Ni film of 18 μm thick onto the exposed Cu surface resulting in isolated reaction-well barriers 1704. The solution formula and plating conditions are as follows: NiSO₄.6H₂O: 300 g/l, NiCl₂.6H₂O: 30-40 g/l, Boric Acid: 40 g/l, Sodium Saccharin: 2-5 g/l, Butynediol (2-Butyne-1,4-diol): 100 mg/l, Sodium lauryl sulfate: 50 ppm, pH: 3.0-4.2, Current density: 10 A/dm², Temperature: 50° C. The photoresist film 1703 was then stripped. Cu film was etched using a HNO₃:H₃PO₄:CH₃COOH=0.5:50.0:49.5 (volume) solution and Cr film was etched using a HCl:H₃PO₄:CH₃COOH=5:45:50 (volume) solution activated by an aluminum stick. A spin-on glass film was then coated onto the sample surface to form an SiO₂ film 1705. FIG. 17B shows a close-up photograph of the resulting microarray or biochip showing the isolated reaction-wells.

### Example VII

### Patterned Non-Wetting Film Generation of Isolated Reaction-Wells

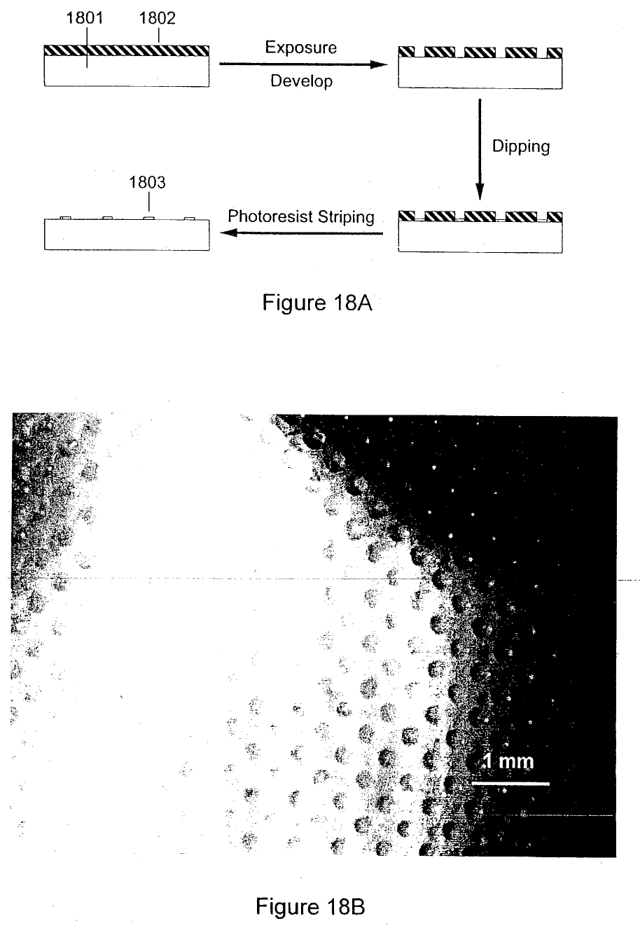

This example illustrates that the present invention contempates generating microarrays and biochips containing isolated reaction-wells formed by droplets on a glass surface by the use of patterned with non-wetting films. FIG. 18A schematically illustrates a fabrication procedure for coating a patterned non-wetting film on a glass solid substrate 1801. First, a glass solid substrate 1801 was thoroughly cleaned in a warm H₂SO₄:H₂O as a 1:1 (volume) solution. Then, substrate 1801 was spin-coated with a positive photoresist of about 2.7 pm thick. The photoresist film 1802 was exposed to UV light using a photomask aligner and developed. In this example, a photomask containing a matrix of circular dots was used and, therefore, the same pattern was formed in the photoresist film 1802. The patterned glass substrate was dipped into a 1 mM FDTS (1H, 1H, 2H, 2H-perfluorodecyltrichlorosiliane) anhydrous iso-octane solution in a dry box and soaked for at least 10 minutes. Then, glass substrate 1801 was rinsed with iso-octane 2-3 times followed by a thorough water rinse. The photoresist was stripped and a FDTS film was left on the glass of glass solid substrate 1801 as a non-wetting film.

Tests of wetting effects were performed in an enclosed cell to avoid evaporation of volatile solvents. During a test, the cell was filled with a testing solvent or solution and then drained. Tests were made on various organic/inorganic solvents and solutions including CH₂Cl₂, CH₃CN, CH₃OH, CH₃CH₂OH, TCA/CH₂Cl₂ solution, 1₂/tetrahydrofuran-water-pyridine solution, and other solutions involved in oligonucleotide synthesis. Formation of isolated reaction-wells (i.e., droplets) was observed for each testing solvent/solution. FIG. 18B shows a photograph of isolated reaction-wells comprising methanol droplets formed on a patterned non-wetting film glass solid substrate.

### Example VIII

### Nucleotide Dimer Synthesis on a Patterned Glass Solid Substrate using Acid PGRs

These experiments demonstrate the use of the method and instrument of the present invention in making microarrays and biochips comprising oligonucleotides.

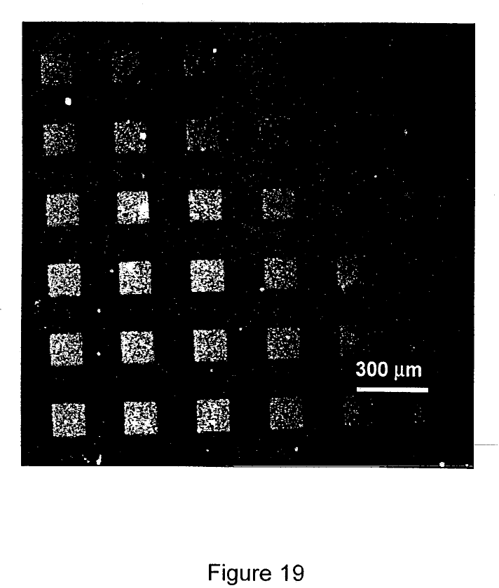

Glass solid substrates containing isolated reaction-wells at specified areas were fabricated as described in Example VI. The glass solid substrates were derivatized with linkers (10% N-(3-triethoxysilylpropyl)-4-hydroxylbutyramide in ethanol) containing free —OH groups (i.e., unprotected). Synthesis on the glass solid substrate was performed using a reactor assembly and an optical system as described in this specification in combination with a DNA synthesizer (Perspective). Oligonucleotide synthesis was accomplished according to the protocol shown in Table 2A. The derivatized glass solid substrate was first contacted with DMT-T phosphoramidite to couple the first protected nucleotide monomer. The multimers were treated, in subsequent steps, with capping and oxidation reagents and washed with CH₃CN before and after each step of the reactions. The glass solid substrate was then treated with an acid PGR (0.4% of 50% triarylsulfonium hexaflurophosphate in CH₂Cl₂) delivered by the synthesizer and exposed to a controlled light pattern (30 s) from a collimated light source at 365 nm and 3 mw of light source intensity (Stanford, Calif.). The glass solid substrate was then extensively washed with CH₃CN. In the illuminated isolated reaction-wells free hydroxyl groups were generated (i.e., via proton-mediated deprotection from acid PGRs). After oxidation and wash steps, the glass solid substrate was contacted by fluorescein-labeled phosphoramidite monomers in a second coupling step. The molecular arrays synthesized were treated with NaOH aqueous solution (0.1 M). Each isolated reaction-well contains a fluorescence labeled dimer that were visualized under a fluoromicroscope (Bio-Rad, Richmond, Calif.)(see FIG. 19).

### Example IX

### Peptide Synthesis on a Glass Solid Substrate

Glass microscope slides or microarray/biochip solid substrates were cleaned with a mixture of H₂SO₄ and H₂O₂ (6:4, v/v) for 30 min at room temperature. After thoroughly washing with deionized water and 95% ethanol, the solid substrates were treated with a mixture of 0.1% aminopropyltriethoxysilane and 2.5% triethoxysilane in 95% ethanol for 30 min. The solid substrates were then washed with ethanol and cured at 110° C. for 20 min. The solid substrates were kept in a dry, N₂-filled chamber until use.

Solid substrates were derivatized with linkers and then contacted with BOC-protected amino acids (0.5 mmol) in 20 mL of DCM/DMF (1:1, v/v) in the presence of DIC (0.5 mmol) and HOBt (0.5 mmol) for 2-4 h at room temperature to form a coupled linker-amino acid multimer. The solid substrates were then washed thoroughly with DCM/DMF (1:1, v/v), DMF, DCM, and DMF, respectively. The remaining free amino reactive terminal groups on non-coupled linkers were permanently blocked with 50% acetic anhydride in DMF for 30 min, followed by washing with solvents.

The multimer BOC protection group was removed by either i) the conventional method, wherein the solid substrate is contacted with 50% TFA in DCM for 1 h at room temperature followed by a thorough washing; or ii) the acid PGR method, wherein the solid substrate is contacted with 10% SSb in DCM and illuminated with a controlled light pattern of UV light for 20 minutes which initiated the deprotection reaction by production of acid PGRs. Following illumination, the deprotection reaction continued for an additional 20 minutes. Subseqnently, the solid surface was washed with solvents. Following the deprotection step both sets of samples (i.e., conventional acid or acid PGR deprotection) were neutralized by 5% DIEA in DMF for 5 min, followed by solvent washing. The coupling, capping, and deprotection cycle was repeated until an oligopeptide of the desired length was obtained. The removal of any protection groups on amino acid side-chains was performed following the instructions of the vendor (Calbiochem-Novabiochem).

### Example X

### Peptide Synthesis on a Microarray or BioChip

Microarray/biochip glass solid substrates (22 mm×75 mm) containing 144 isolated reaction-wells with diameter of 600 μm and depth of 30 μm confined in a 1.4×1.4 cm² area were prepared by novel semiconductor manufacturing techniques. Srivannavit, O., Ph.D. Thesis, University of Michigan (2002). The surface outside the isolated reaction-wells was derivatized with a solution of (heptadecafluoro-1,1,2,2-tetrahydrodecyl)triethoxysilane in isooctane (10 mM, 50 mL) overnight at room temperature under gentle shaking.

The microarray/biochip solid substrate was placed into cartridge reactor assembly. The coupling and capping steps were achieved by injection of reagent solutions, as explained in Example IX, into the cartridge reactor assembly. The deprotection at the desired locations was performed by using 2.5% SSb (mixed with perylene, 1:0.1) activated by illumination of isolated reaction-wells with a controlled light pattern generated from a laser beam for approximately 30 minutes. Perylene was included as a sensitizer for SSb, because the light adsorption peak for SSb precursor is centered at 365 nm whereas the wavelength of our light source is 405 mm. Although it is not necessary to understand the exact mechanism of the present invention, SSb sensitization by perylene is believed to be primarily due to electron transfer to SSb precursor. Crivello et al., Dye-Sensitized Photoinitiated Cationic Polymerization, The System: Perylene-Triarylsulfonium Salts, J. Polym. Sci. 17:1059-1065 (1979). Following illumination, the deprotection reaction continued for an additional 4 min, after which the acid was neutralized by injection of a 5% DIEA solution. Prior to another illumination cycle, the cartridge reactor assembly was flushed with a series of washing solvents. In this particular example, the deprotection and neutralization steps were repeated five times at the same location to obtain a high level of deprotection.

### Example XI

### Fluorescence Labeling and Detection Oligopeptides

Two types of fluorophores, 5,6-carboxyfluorescein (FAM) and dansyl chloride (dns) are capable of binding with the amino functional group. FAM was used to detect free amino reactive terminal groups present on the solid substrate, and dns was used for the signaling of a synthesized metal-binding peptide.

To detect the free amino reactive terminal groups, oligopeptides attached to a solid substrate were treated with a solution of FAM ester containing 5 μM FAM, HOBt (0.0338 g), and DIC (100 μL) in DMF for 4 h at room temperature. After thorough washing with DMF and ethanol, the solid substrate was then covered with Vectrashield mounting medium (H-1000) to improve the signal/noise ratio for 1 h. Thereafter, the solid substrate was placed under a cooled CCD camera and fluoroescent was generated by an excitation wavelength of 488 nm, and the emission wavelength was detected at 520 nm. Using the emission wavelength data, fluorescence images were acquired, processed, and analyzed using PMIS Image Processing Software program.

5-dimethylaminonaphthalene-1-sulfonyl chloride (dansyl chloride, dns) generated fluorescence was also performed by contacting the solid substrate with a mixture of dns (1.25 mmol) and triethylamine (0.125 mmol) in DMF for 4 h at room temperature. The fluorescence detection was done in the same manner as previously described for FAM with the exception that the excitation wavelength was 365 nm.

### Example XII

### Pb²⁺ and As³⁺ Ion Binding Assay of Oligopeptides

For the Pb²⁺ ion assay, solid substrates comprising oligopeptides were washed with a buffer solution containing 100 mM HEPES (pH 7.1) and 10% methanol. Samples were then treated with 0.1 mM lead chloride in buffer overnight at room temperature. After washing with buffer solution and methanol the solid substrates were left to dry in an inert atmosphere in the dark.

For the As³⁺ ion assay, the assay was performed by the same procedure as the Pb²⁺ assay but the buffer was 10 mM sodium acetate (pH 4.0) containing 100 mM NaCl and 0.1 mM sodium arsenite (NaAsO₂) was the metal ion solution.

### Example XIII

### Oligopeptide Immunoassay using Residues 20-25 Of Human p53

Solid substrates comprising oligopeptids were washed with PBS (phosphate-buffered saline, 25 mM NaH₂PO₄, 125 mM NaCl, pH 7.4), PBST (PBS, 0.2% (V/V) Tween 20), and PBS, respectively. Next, the solid substrates were then incubated with mouse MAb DO-1, which recognizes amino acid residue 20-25 of human tumor suppressor protein p53, in PBS at room temperature for 2 h. After washing with PBST and PBS, samples were treated with FITC-tagged secondary rabbit anti-mouse immunoglobulin (IgG) overnight at 4° C. Following washing with PBST and PBS, fluorescence of the solid substrate was measured.

We claim:

1. A method, comprising:
a) providing;
i) a plurality of protected polypeptides attached to a substrate, each of said protected polypeptides comprising at least one protecting group, said substrate configured so as to comprise of a plurality of isolated reaction sites;
ii) a mixture comprising triarylsulfonium antimonyhexafluoride; and
iii) a laser;
b) contacting one or more of said isolated reaction sites with said mixture to create one or more contacted reaction sites; and
c) illuminating one or more said contacted reaction sites with said laser under conditions such that said protecting group of at least one protected polypeptide is removed so as to create a deprotected polypeptide.

2. The method of claim 1, wherein said mixture further comprises dichloromethane.

3. The method of claim 2, wherein said mixture further comprises perylene.

4. The method of claim 1, wherein said substrate prior to step (a) was derivatized.

5. The method of claim 4, wherein said substrate is derivatized with triethoxysilane.

6. The method of claim 1, wherein said protecting group is tert-butoxycarbonyl.

7. The method of claim 1, further comprising step d) contacting said deprotected polypeptide with a first protected monomer under conditions such that said first protected monomer is coupled to said polypeptide to create a first extended polypeptide, said first protected monomer comprising a protecting group.

8. The method of claim 7, further comprising step e) illuminating said first extended polypeptide with said laser under conditions such that said protecting group of said first protected monomer is removed so as to create a second extended polypeptide.

9. The method of claim 8, further comprising step (f) contacting said second extended polypeptide with a second protected monomer under conditions such that said second protected monomer is coupled to said second extended polypeptide to create a third extended polypeptide, said third extended polypeptide comprising a protecting group.

10. The method of claim 1, wherein said plurality of isolated reaction sites of step (a) is selected from the group consisting of reaction wells, reactor assembly cartidges and non-wetting surfaces.

11. A method, comprising:
a) providing;
i) a plurality of protected first monomers attached to a substrate, each of said protected first monomers comprising at least one protecting group, said substrate configured so as to comprise of a plurality of isolated reaction sites;
ii) a mixture comprising trisarylsulfonium antimonyhexafluoride; and
iii) a laser;
b) contacting one or more of said isolated reaction sites with said mixture to create one or more contacted reaction sites; and
c) illuminating one or more said contacted reaction sites with said laser under conditions such that said protecting group of at least one protected first monomer is removed so as to create a deprotected first monomer.

12. The method of claim 11, wherein said mixture further comprises dichloromethane.

13. The method of claim 12, wherein said mixture further comprises perylene.

14. The method of claim 11, wherein said substrate prior to step (a) is derivatized.

15. The method of claim 14, wherein said substrate is derivatized with tertbutoxycarbonyl.

16. The method of claim 11, further comprising step d) contacting said deprotected first monomer with a protected second monomer under conditions such that said protected second monomer is coupled to said first monomer to create a first protected biopolymer, said protected second monomer comprising a protecting group.

17. The method of claim 16, further comprising step e) illuminating said first protected biopolymer with said laser under conditions such that said protecting group of said protected second monomer is removed so as to create a first deprotected biopolymer.

18. The method of claim 17, further comprising step (f) contacting said first deprotected biopolymer with a third protected monomer under conditions such that said third protected monomer is coupled to said first deprotected biopolymer to create a second protected biopolymer, said second protected biopolymer comprising a protecting group.

19. The method of claim 11, wherein said monomer comprises an amino acid.

20. A method, comprising:
a) providing;
i) a plurality of protected linkers attached to a substrate, each of said protected linkers comprising at least one protecting group, said substrate configured so as to comprise of a plurality of isolated reaction sites;
ii) a mixture comprising triarylsulfonium antimonyhexafluoride; and
iii) a laser;
b) contacting one or more of said protected linkers with said mixture to create one or more contacted protected linkers; and
c) illuminating one or more said protected linkers with said laser under conditions such that said protecting group of at least one of said protected linkers is removed so as to create a deprotected linker.

21. The method of claim 20, wherein said mixture further comprises dichloromethane.

22. The method of claim 21, wherein said mixture further comprises perylene.

23. The method of claim 20, wherein said substrate prior to step (a) is derivatized.

24. The method of claim 23, wherein said substrate was derivatized with tertbutoxycarbonyl.

25. The method of claim 20, further comprising step d) contacting said deprotected linker with a first protected monomer under conditions such that said first protected monomer is coupled to said deprotected linker to create a first multimer, said first protected monomer comprising a protecting group.

26. The method of claim 25, further comprising step e) illuminating said first multimer with said laser under conditions such that said protecting group of said first protected monomer is removed so as to create a deprotected first multimer.

27. The method of claim 26, further comprising step (f) contacting said deprotected first multimer with a second protected monomer under conditions such that said second protected monomer couples with said deprotected first monomer to create a second multimer, said second protected monomer comprising a protecting group.


SEQUENCE LISTING (12 sequences)

SEQ ID NO: 1 - protein, 4 aa, Artificial Sequence
  Synthetic
ECEE

SEQ ID NO: 2 - protein, 4 aa, Artificial Sequence
  Synthetic
EEEE

SEQ ID NO: 3 - protein, 4 aa, Artificial Sequence
  Synthetic
VDAE

SEQ ID NO: 4 - protein, 4 aa, Artificial Sequence
  Synthetic
TYAQ

SEQ ID NO: 5 - protein, 4 aa, Artificial Sequence
  Synthetic
ADSQ

SEQ ID NO: 6 - protein, 6 aa, Artificial Sequence
  Synthetic
SDLHKL

SEQ ID NO: 7 - protein, 6 aa, Artificial Sequence
  Synthetic
DSLHKL

SEQ ID NO: 8 - protein, 6 aa, Artificial Sequence
  Synthetic
SGLHKL

SEQ ID NO: 9 - protein, 4 aa, Artificial Sequence
  Synthetic
CCCC

SEQ ID NO: 10 - protein, 4 aa, Artificial Sequence
  Synthetic
GGGG

SEQ ID NO: 11 - protein, 6 aa, Artificial Sequence
  Synthetic
DSLGKL

SEQ ID NO: 12 - protein, 5 aa, Artificial Sequence
  Synthetic
LFGGY